(19) United States
(12) Patent Application Publication
(10) Pub. No.: US 2016/0200769 A1
(43) Pub. Date: Jul. 14, 2016

(54) Compound, Transitmycin, Effective Against Bacterial and Viral Pathogens

(71) Applicant: INDIAN COUNCIL OF MEDICAL RESEARCH, New Delhi (IN)

(72) Inventors: Vanaja Kumar, Tamil Nadu (IN); Mukesh Doble, Tamil Nadu (IN); Balagurunathan Ramasamy, Tamil Nadu (IN); Suresh Ganesan, Tamil Nadu (IN); Radhakrishnan Manikkam, Tamil Nadu (IN); Luke Elizabeth Hanna, Tamil Nadu (IN); Soumya Swaminathan, Tamil Nadu (IN); Selvakumar Nagamiah, Tamil Nadu (IN)

(21) Appl. No.: 14/912,123

(22) PCT Filed: Jan. 9, 2014
(86) PCT No.: PCT/IN2014/000020

§ 371 Date: Feb. 15, 2016

(30) Foreign Application Priority Data
Aug. 16, 2013 (IN) 2435/DEL/2013

(51) Int. Cl. C07K 11/02; A61K 35/74; C12P 21/00; A61K 38/15

(52) U.S. Cl. CPC C07K 11/02; A61K 38/15; A61K 35/74; C12P 21/00

(57) ABSTRACT

Provided herein is a compound represented by Formula (I) (Transitmycin) effective against bacterial and viral pathogens.

### FIELD OF THE INVENTION:

The invention relates to a compound effective against bacterial and viral pathogens.

### BACKGROUND OF THE INVENTION:

The incidence of infections caused by drug resistant bacteria continues to increase and remains a serious threat to human health (Asolkar et al, 2010). Disease causing bacteria such as Mycobacterium tuberculosis, Staphylococcus aureus, Escherichia coli, Pseudomonas aeruginosa gradually develop resistance to drugs. Out of all these the drug resistance developed by Mycobacterium tuberculosis against the commonly used antibiotics is of major concern. Tuberculosis remains one among the leading causes of infectious disease worldwide. One third of the world population is infected with Mycobacterium tuberculosis and hence at risk of developing active TB (Boogoard et al, 2009).

The current first line TB regimen is more than 40 years old and consists primarily of rifampicin and isoniazid. These antibiotics are effective in active drug susceptible TB, provided that patients complete the course of treatment. However, there is a poor patients' compliance due to the cost of drugs, adverse effects, the long time required for completion of treatment (6-12 months) and the required number of drug doses. Non-compliance has contributed to the emergence of multi drug resistant (MDR) and extensively drug resistant (XDR) TB strains. MDR TB (strains resistant to isoniazid and rifampicin) often takes longer time to treat with second line drugs. XDR-TB (MDR TB resistant to second line drugs including fluoroquinolones and any one of the injectable drugs such as capreomycin, kanamycin and amikacin) is virtually incurable. Furthermore, HIV/AIDS antiretroviral therapies are not always compatible with the current TB regimen because of shared drug toxicities and drug interactions (Rivers and Mancera, 2008). In this context, there is an urgent need for developing novel antiTB drugs with less toxic side effects, improved pharmacokinetic properties with extensive and potent activity against resistant strains and to reduce the total duration of treatment (De Sousa, 2006).

Actinomycetes are the most economically valuable prokaryotes which are well known to produce chemically diverse metabolites with wide range of biological activities. It has been estimated that about half of the microbial bioactive metabolites notably antibiotics, antitumor agents, immuno suppressives and enzyme inhibitors have been isolated from actinomycetes (Balagurunathan and Radhakrishnan, 2010). Recently the rate of discovering new compounds from terrestrial actinomycetes has decreased but the rate of re-isolation of known actinomycetes and antibiotics is on the increase. This has led researchers to explore unique and extreme habitats such as marine environment for potentially new biosynthetic diversity. Marine actinomycetes are the promising source for secondary metabolites (Lam, 2006). In the past 10 years, 659 marine bacterial compounds have been described in which 256 compounds have originated from actinomycetes (Williams, 2008).

From the discovery of streptomycin from Streptomyces griseus, actinomycetes derived antibiotics are still in use for the treatment of tuberculosis. Due to the emergence of MDR and XDR TB cases, search for novel antibiotics is still continuing.

### OBJECTS OF THE INVENTION:

The primary objective of the invention is to provide a compound which is effective against bacterial and viral pathogens.

Another objective of the invention is to provide a process of preparing the compound.

Yet another objective of the invention is to provide a novel strain of Actinomycetes which produces the chemical compound having activity against bacterial and viral pathogens.

These and other objects of the invention will be apparent from the ensuing description, when read in conjunction with the accompanying drawings.

### SUMMARY OF THE INVENTION:

This invention relates to a compound represented by formula (I)

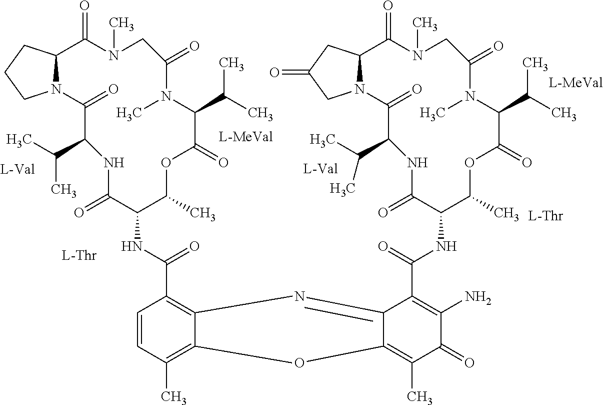 [SMILES: Cc1c2oc3c(C)ccc(C(=O)N[C@@H]4C(=O)N[C@@H](C(C)C)C(=O)N5CCC[C@H]5C(=O)N(C)CC(=O)N(C)[C@@H](C(C)C)C(=O)O[C@@H]4C)c3nc-2c(C(=O)N[C@@H]2C(=O)N[C@@H](C(C)C)C(=O)N3CC(=O)C[C@H]3C(=O)N(C)CC(=O)N(C)[C@@H](C(C)C)C(=O)O[C@@H]2C)c(N)c1=O]

### BRIEF DESCRIPTION OF THE ACCOMPANYING DRAWINGS:

FIG. 1 : RP HPLC of the. Ethyl acetate extract of crude R2

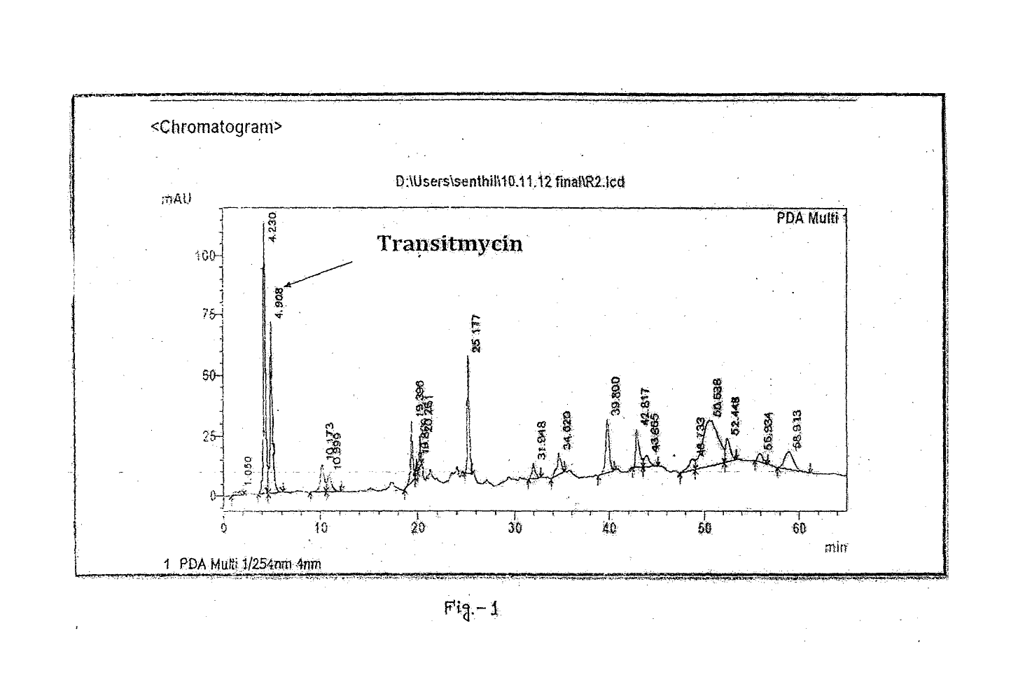

FIG. 2: RP HPLC of Chromatogram of Trasitmycin

FIG. 3 : UV/Vis Spectrum of Transitmycin in methanol

FIG. 4: Circular Dichroism Spectrum of Trasitmycin in methanol

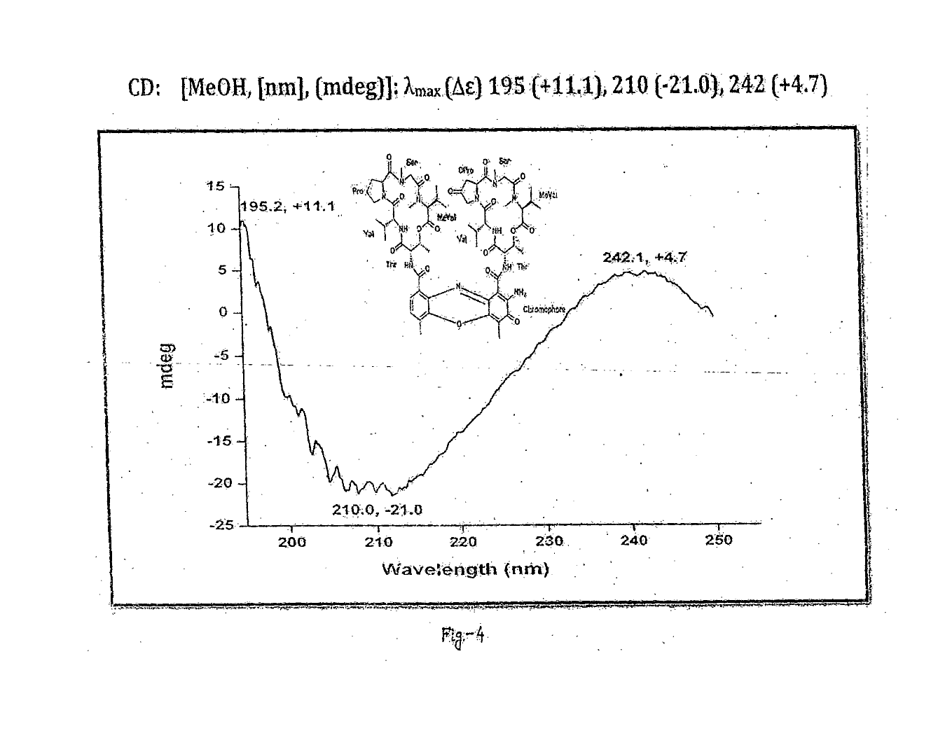

FIG. 5: IR Spectrum of Transitmycin

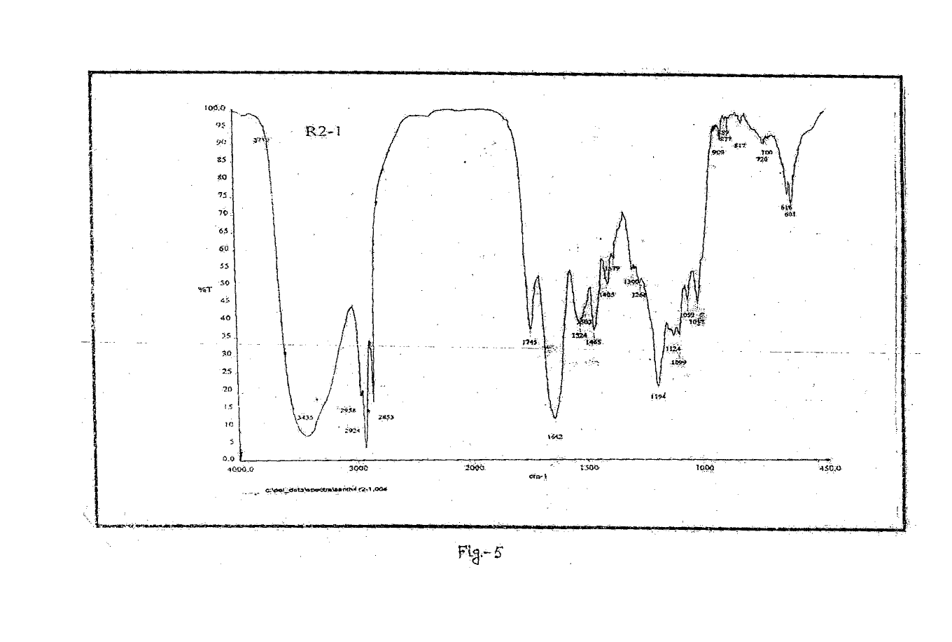

FIG. 6: ¹H-NMR(500 MHz, CDCl₃) Spectrum of Transitmycin

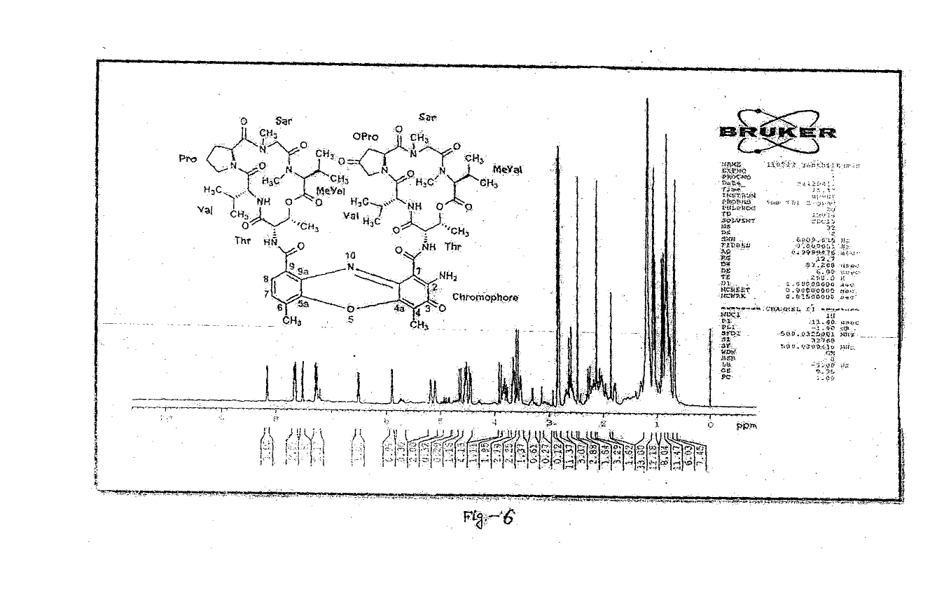

FIG. 7: ¹³C-NMR(125 MHz, CDCl₃) Spectrum of Transitmycin

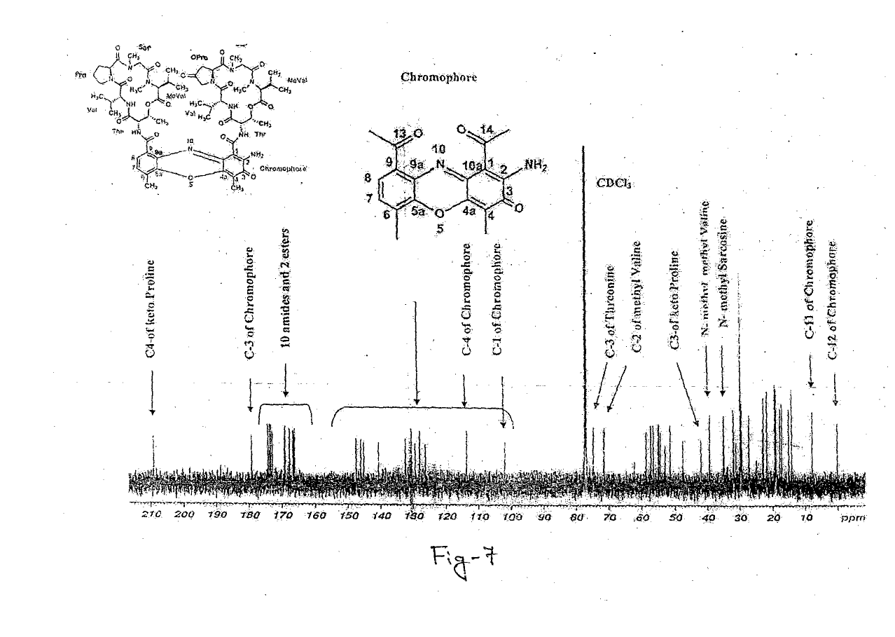

FIG. 8: DEPT135(125 MHz, CDCl₃) spectrum of Transitmycin

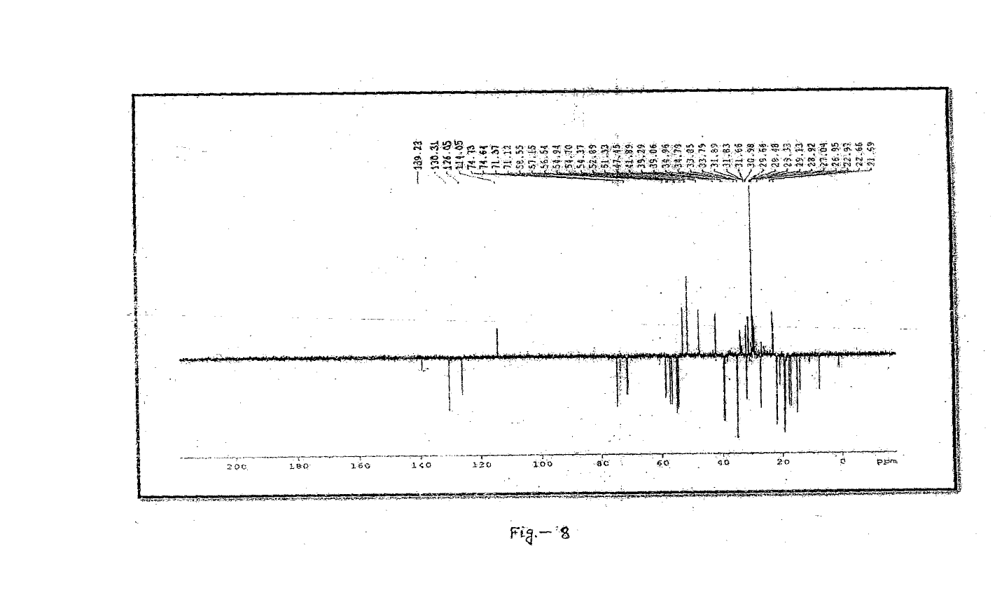

FIG. 9: COSY (500 MHz) Spectrum of Transitmycin

FIG. 10: DQF-COSY (500 MHz) Spectrum of Transitmycin

FIG. 11: HMBC (500 MHz) Spectrum of Transitmycin

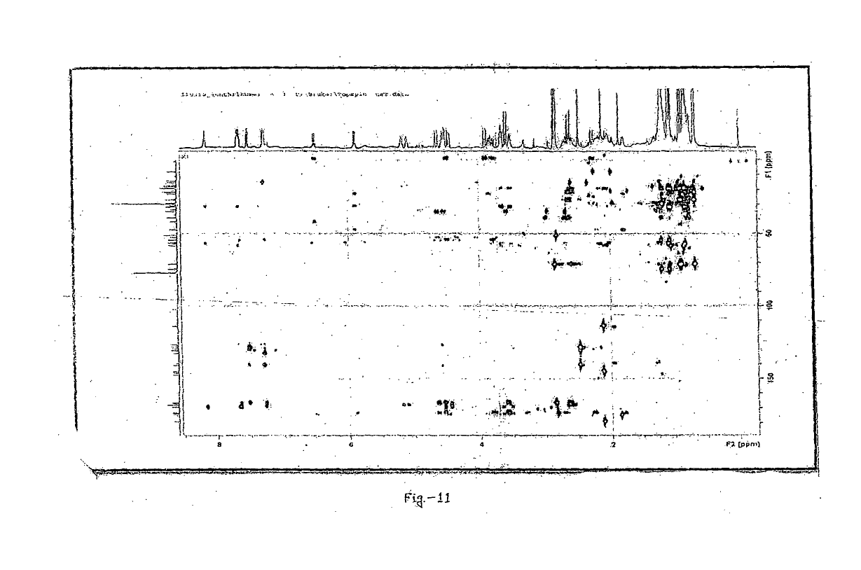

FIG. 12: HSQC (500 MHz) Spectrum of Transitmycin

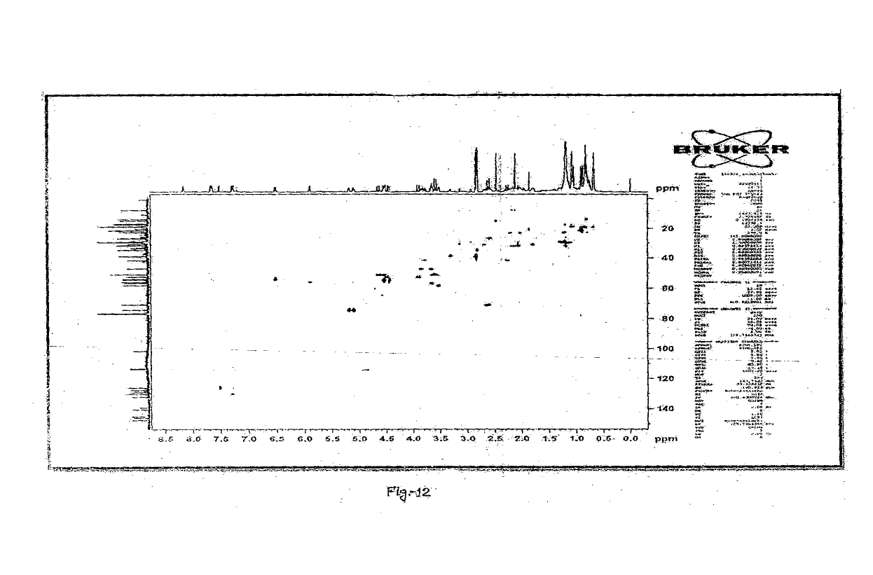

FIG. 13: TOCSY (500 MHz) Spectrum of Transitmycin

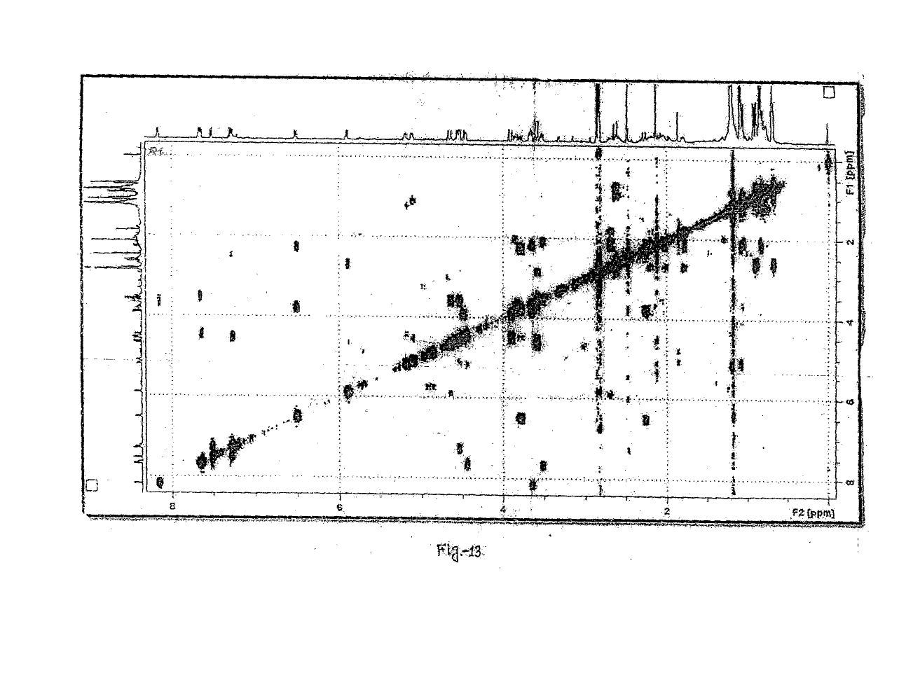

FIG. 14: NOESY (500 MHz) Spectrum of Transitmycin

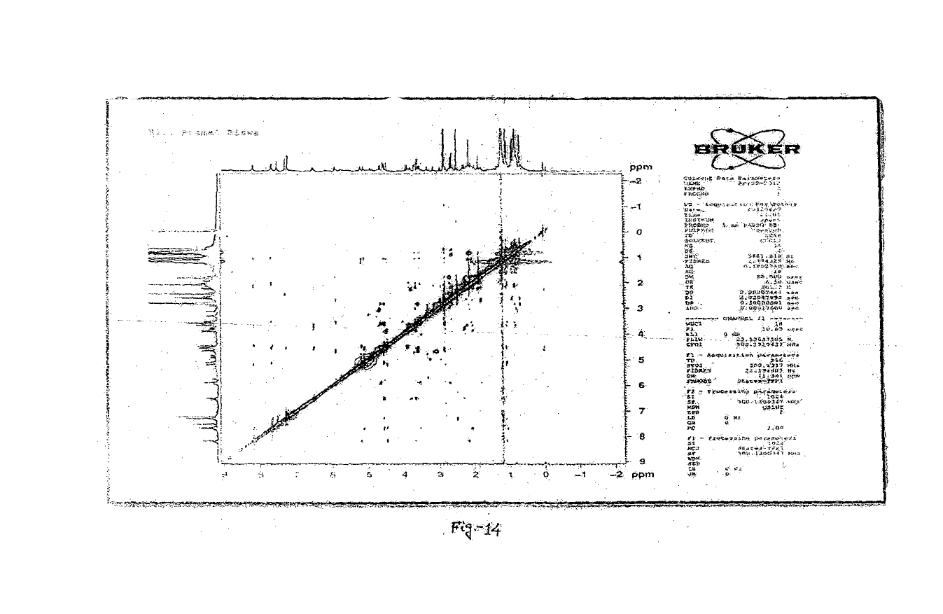

FIG. 15: ROESY (500 MHz) Spectrum of Transitmycin

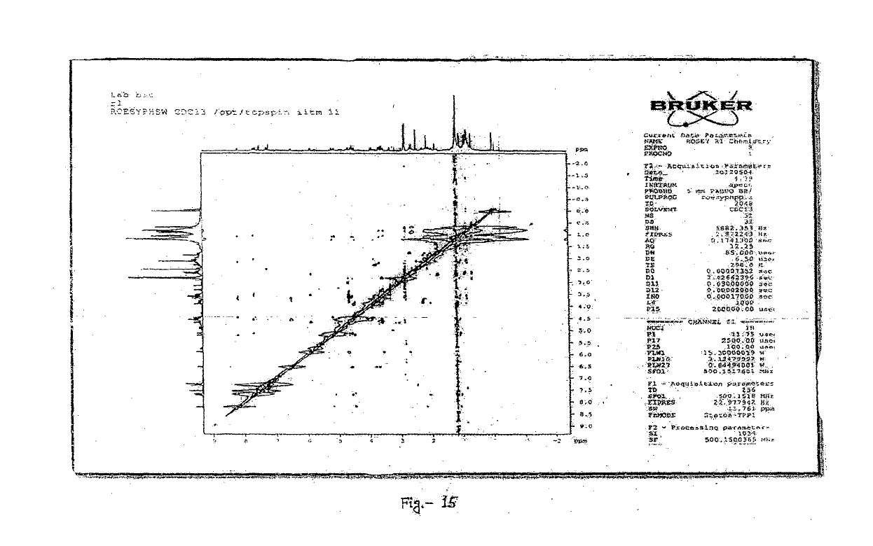

FIG. 16: MALDI-TOF MS Spectrum of Transitmycin (Positive mode)

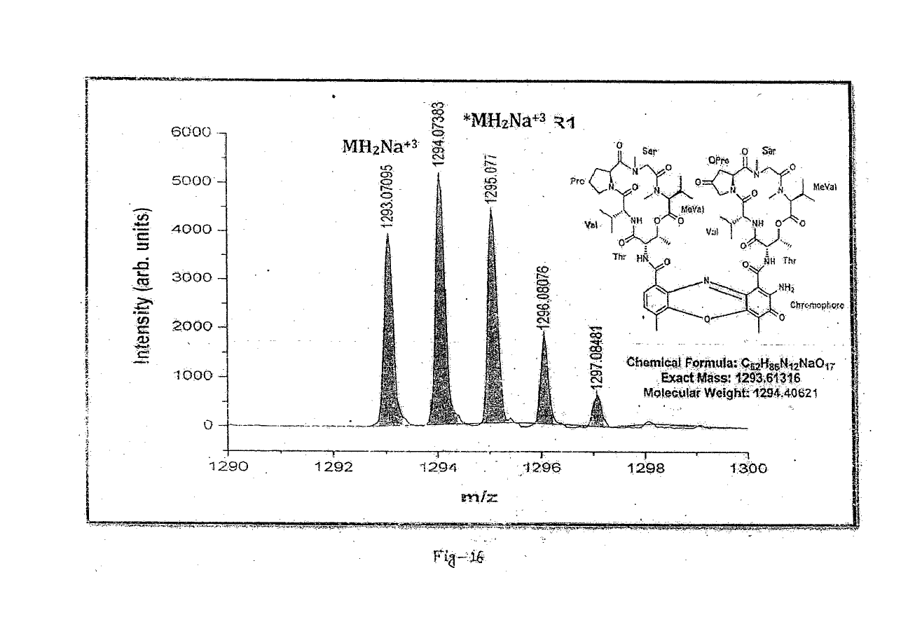

FIG. 17: MALDI-TOF MS Spectrum of Transitmycin (Negative mode)

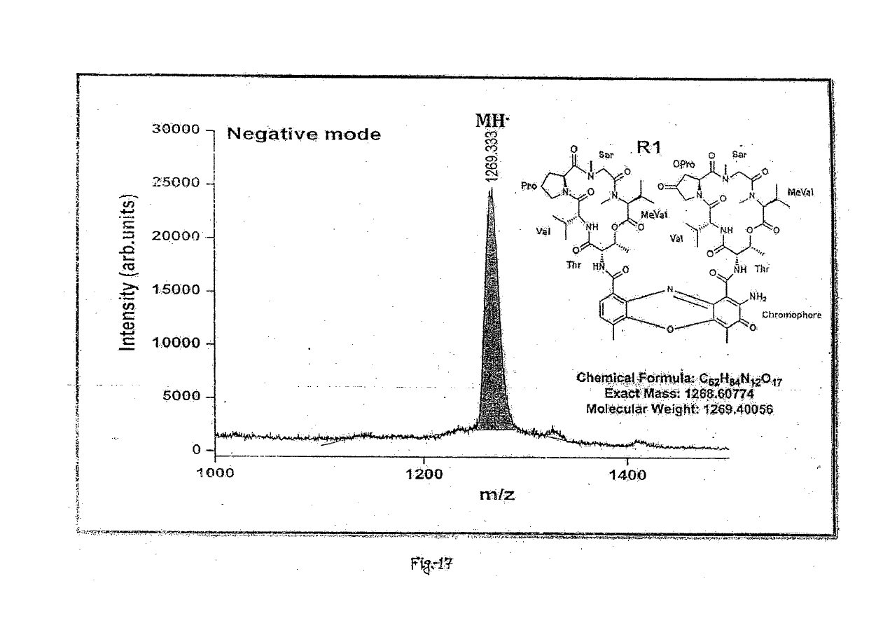

FIG. 18: ESI-MS Spectrum of Transitmycin (Positive mode)

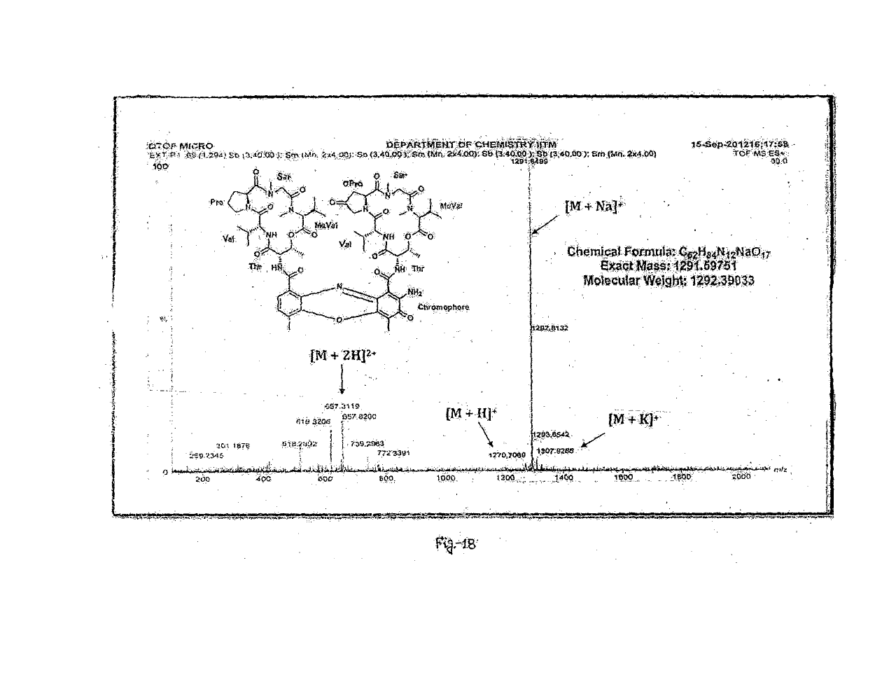

FIG. 19: Expansion of ESI-MS Spectrum of Transitmycin (Positive mode)

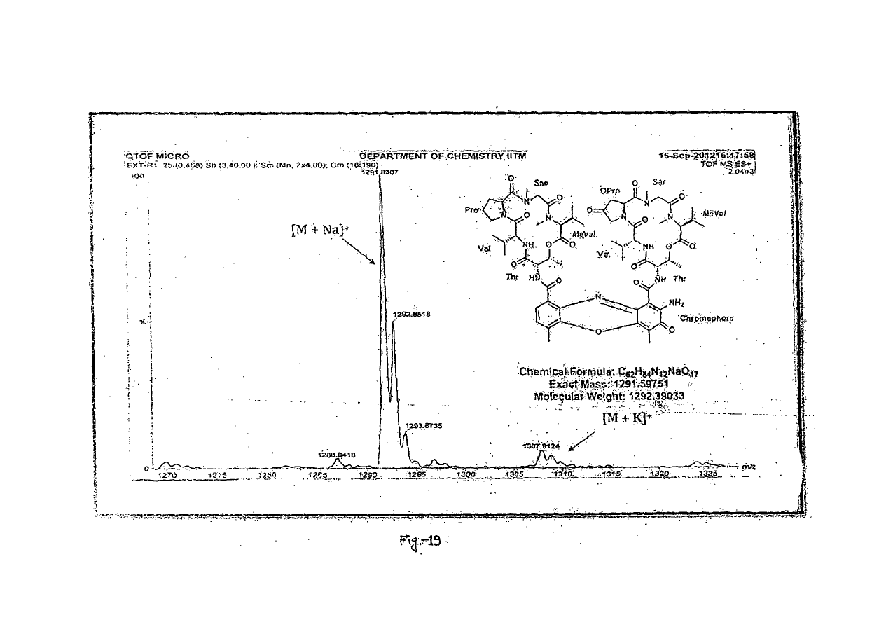

FIG. 20: LC-ESI-MS Spectrum of Transitmycin (Positive mode)

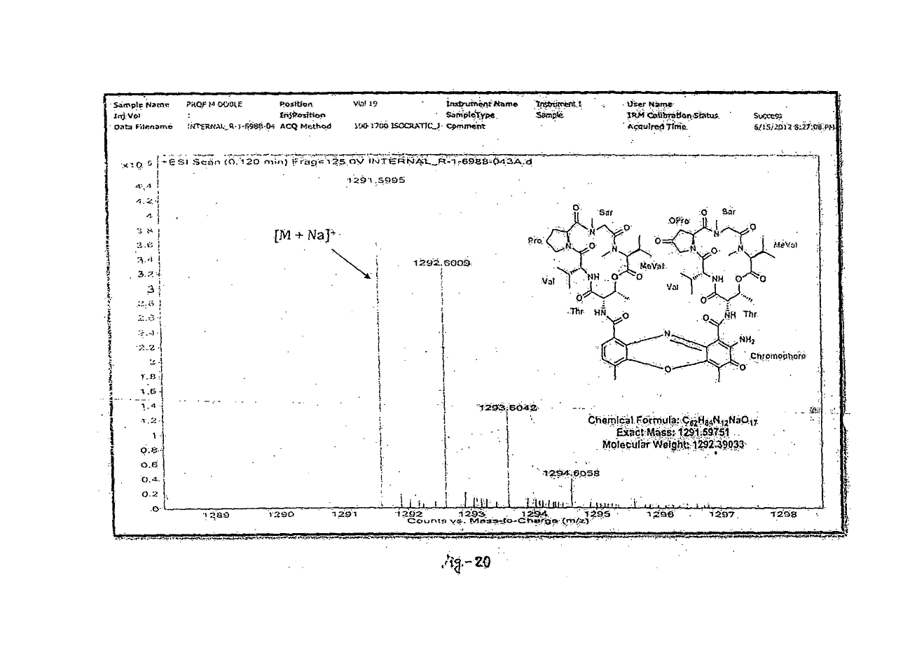

FIG. 21: Expansion of LC-ESI-MS Spectrum of Transitmycin (Positive mode)

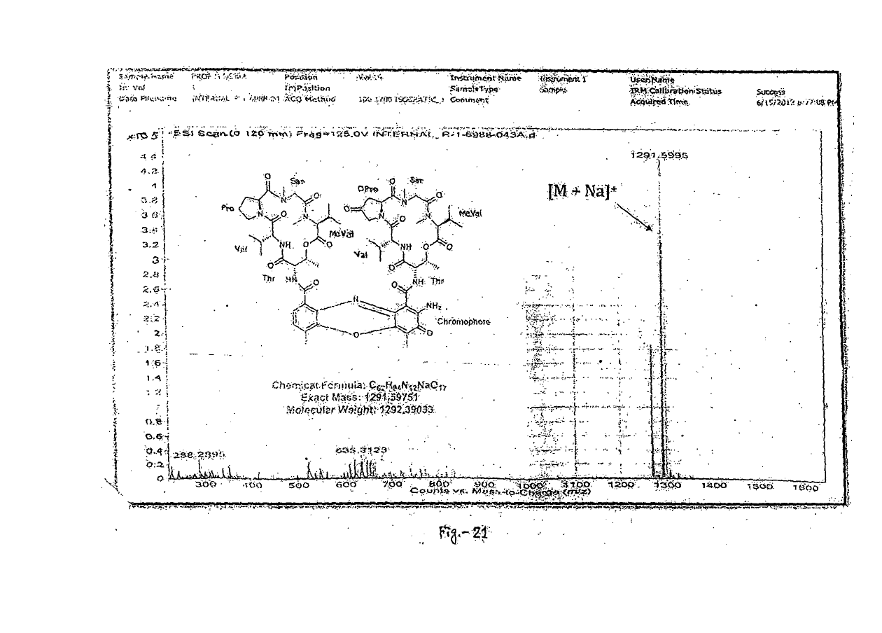

FIG. 22: LC-ESI-MS Spectrum of Transitmycin (Positive mode)

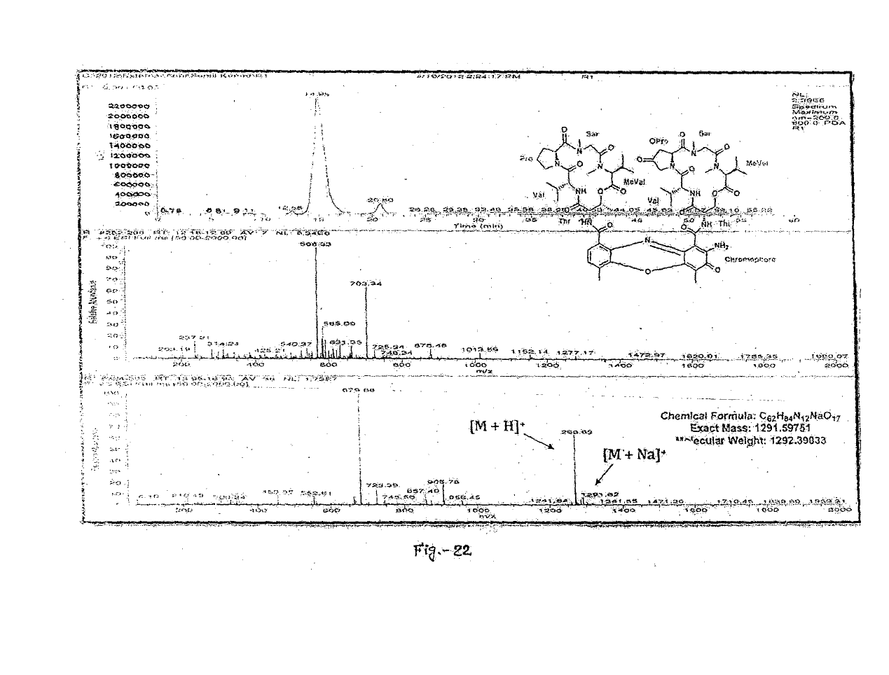

FIG. 23: EI-MS Spectrum of Transitmycin

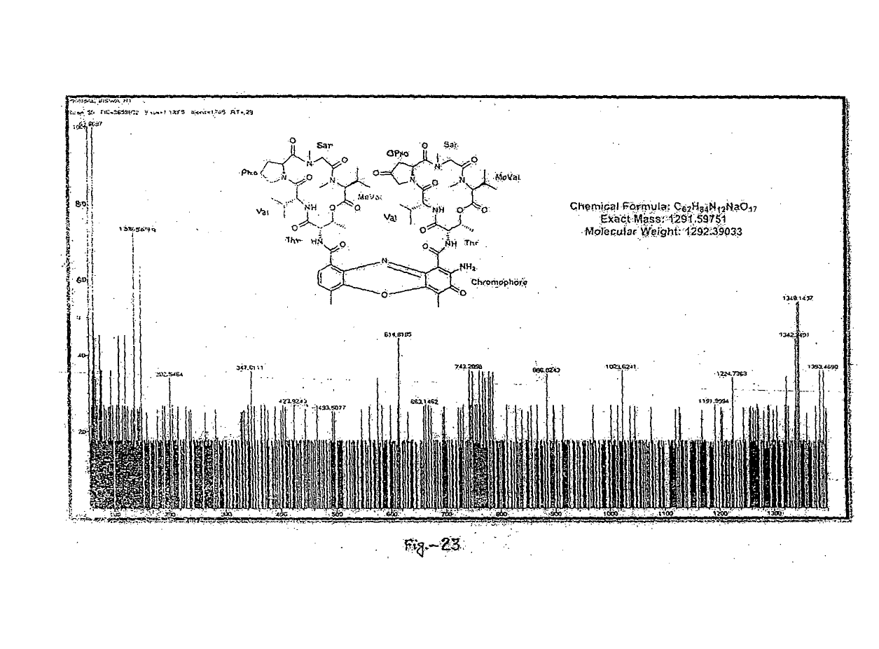

FIG. 24: HPLC analysis of L-FDAA (Marfey's HMBC (500 MHz) Spectrum of Transitmycin

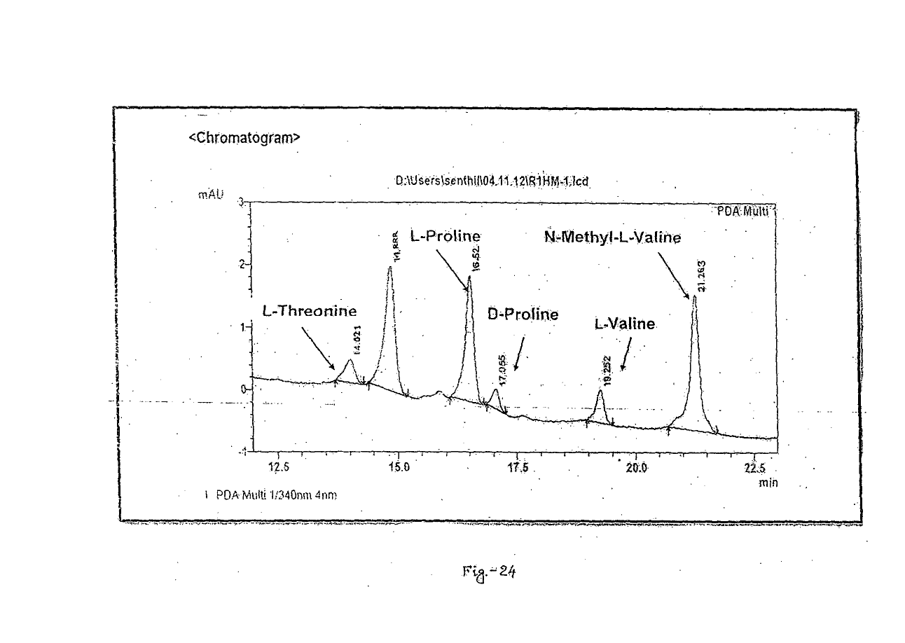

FIG. 25: HPLC analysis of Standard L-FDAA-D-Valine

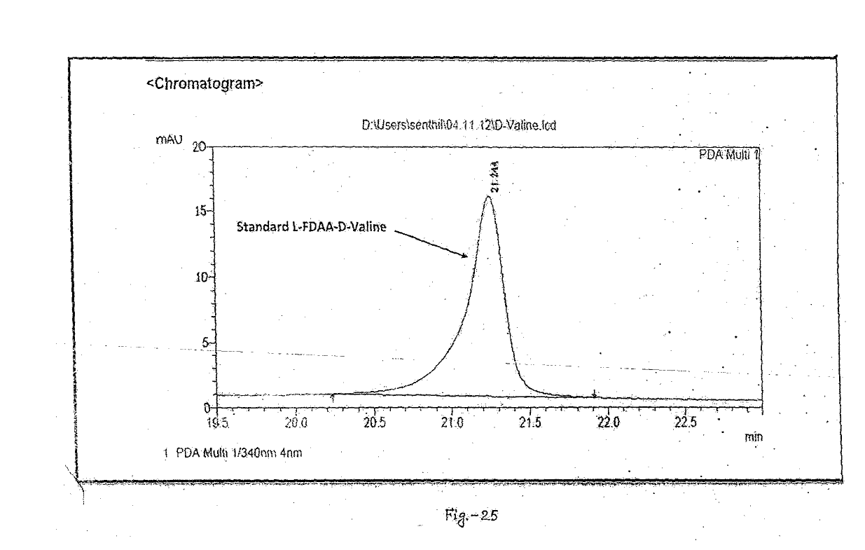

### DETAILED DESCRIPTION OF THE INVENTION:

According to this invention is provided a compound Transitmycin, represented by formula (I)

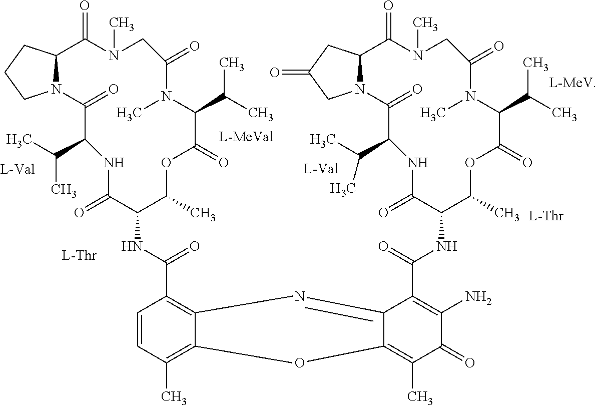 [SMILES: Cc1c2oc3c(C)ccc(C(=O)N[C@@H]4C(=O)N[C@@H](C(C)C)C(=O)N5CCC[C@H]5C(=O)N(C)CC(=O)N(C)[C@@H](C(C)C)C(=O)O[C@@H]4C)c3nc-2c(C(=O)N[C@@H]2C(=O)N[C@@H](C(C)C)C(=O)N3CC(=O)C[C@H]3C(=O)N(C)CC(=O)N(C)[C@@H](C(C)C)C(=O)O[C@@H]2C)c(N)c1=O]

or a derivative thereof.

The chemical structure of the compound is elucidated based on its spectral data. The molecular formula of the compound is established as C₆₂H₈₄N₁₂O₁₇

The compound of the invention is effective against bacterial pathogens such as Mycobacterium tuberculosis, Bacillus subtilis, Bacillus pumilus, Bacillus cereus, Staphylococcus aureus, and Acinetobacter baumanii. The compound is also effective against viral pathogens such as Human Immuno Deficiency Virus (HIV). The compound is effective against multiple drug resistant and extensively drug resistant strains of Mycobacterium tuberculosis. The compound also shows good activity against SHRE (Streptomycin, Isoniazid, Rifampicin, and Ethambutol) sensitive and SHRE resistant strains of Mycobacterium tuberculosis.

The invention further provides a composition comprising the compound of formula (I) along with pharmaceutically acceptable additives, excipients and adjuvants. The composition can be formulated in various forms such as liquid, solid, powder and lozenges.

Suitable excipients are mixed in composition to improve the stability of the composition. Such excipients are selected from the group comprising liquid or solid carrier, disintegrator, coating agents etc. The excipients are further useful for improving efficacy of composition for controlling the bacterial and viral pathogens. The pharmaceutically acceptable additives and excipients are selected from the group comprising glycerol, lactic acid, poly ethylene glycol (PEG), salts such as KCl; cationic surfactants, anionic surfactants and natural surfactants, lactose, sucrose, dextrose, sorbitol and mannitol; dextrins; polycarboxylic acids, chitosan, vitamin C; polyethylene glycols, polyvinyl pyrrolidone, benzyl alcohol and polyvinyl acetate.

The invention further provides use of compound of formula (I) against bacterial and viral pathogens. The invention further provides a method of using compound of formula (I) against bacterial and viral pathogens.

The invention further provides a process of preparing the compound of formula (I), said process comprising the steps of:

(i) inoculating Actinomycetes strain MTCC 5597 onto a suitable agar based media;

(ii) incubating the agar plate(s) at a temperature of 20° C. to 40° C. for a period of 3 to 10 days and obtaining mycelial growth;

(iii) removing the mycelial growth from the agar plate(s) and obtaining the agar medium containing the compound of formula (I);

(iv) optionally cutting the media into pieces;

(v) adding the media pieces into a suitable solvent;

(vi) incubating the media dissolved in the solvent at a temperature of 23° C. to 30° C. for a period of 3 to 18 hours and extracting the compound of formula (I);

(vii) collecting the solvent part and concentrating the same;

(viii) obtaining the concentrate containing the compound of formula (I); and

(ix) purifying the compound.

The media for the growth and inoculation is selected from the group comprising yeast extract malt extract agar, glycerol asparagine agar, oatmeal agar, czapek's dox agar and tyrosine agar

In one of the preferred embodiments, the agar plates are incubated fora period of 7 days at a temperature of 28° C. which is an ideal temperature for the growth of Actinomycetes. After obtaining sufficient growth, the mycelia are removed from the medium. The compound of the invention is secreted extracellularly by the Actinomycete strain. Therefore, the compound is easily extracted from the medium used for the growth of the Actinomycete.

After removing the mycelia from the media, the media is dissolved in a suitable solvent for the extraction of the compound of formula (I). The media can be cut into small pieces for easy and better dissolution.

The solvent used for dissolving the media for the purpose of ‘extraction of the compound is an organic solvent. Preferably, such a solvent is selected from the group comprising methanol, chloroform, dichloromethane, diethyl ether, ethyl acetate and n-hexane.

It is preferred that the media dissolved in the solvent is incubated for a period of 24 hours and at a temperature of 28° C.

The concentrate obtained in step (viii) of the process is either directly subjected to the process of purification or is stored at a suitable temperature for further usage.

The preferred temperature for the purpose of storing the concentrate is 4° C. to 25° C. More preferably, the temperature for the purpose of storing the concentrate is 4° C.

The purification of the compound is done by various methods such as chromatography or crystallization methods. Chromatography involves methods such as thin layer chromatography and column chromatography. Column chromatography is useful for large scale production of the compound. Crystallization involves methods such as single solvent recrystallization, multi solvent recrystallization.

The invention further provides an Actinomycetes strain with accession no MTCC 5597. The strain is useful for'producing the compound of formula (I). The Actinomycetes strain of the invention is isolated from coral reef marine ecosystem of, Rameswaram, Tamil Nadu, India.

The invention also provides a biologically active agent comprising a compound of formula(I), wherein the agent is effective against bacterial and viral pathogens.

The invention also provides a kit comprising a compound of formula (I) along with an instruction manual. The kit of the invention may also comprise the Composition along with an instructions manual.

The invention is illustrated further by the following examples which are only meant to illustrate the invention without intending to limit the scope thereof. The embodiments which may be apparent to a person skilled in the art are deemed to fall within the scope of the present invention.

### EXAMPLE 1

### Sample Collection and Isolation of Actinomycete Strain of the Invention

i. Collecting sediment samples from Coral reef ecosystem of Rameswaram, South. India;

ii. Drying the sediment sample at room temperature for five days;

iii. Keeping the sample at 55° C. in hot air oven for 10 minutes;

iv. Serially diluting the sediment sample using sterile distilled water;

v. Plating the diluted sample on nalidixic acid (20 μg/ml) and cycloheximide (100 g/ml) supplemented Starch Casein agar prepared in 50% filtered sea water;

vi. Incubating the plates at 28° C. for one month;

vii. Isolating the colonies with actinomycete morphology and subculturing on. YEME (ISP 2) agar medium prepared in 50% seawater;

viii. Maintaining the stock cultures of actinomycete strain YEME agar slants, 30%>glycerol stock as well as in lyophilized form.

Morphologically distinct actinomycete colonies are observed on starch casein agar medium after 5 days of incubation. Actinomycete strain of the invention produced small colonies with powdery consistency, with yellow colour soluble pigment production. Good growth of actinomycete strain is observed on YEME agar medium.

### EXAMPLE 2

### Antibacterial Activity of Actinomycete strain of the Invention—Agar Plug Method

I. Culturing the actinomycete strain on YEME agar medium at 28° C. for 10 days;

II. Preparing the cell suspensions of test bacterial cultures using nutrient broth and Sabouraud Dextrose broth, respectively, and adjusting the turbidity to 0.5 McFarland standards;

III. Inoculating the cell suspensions on Muller Hinton Agar (MHA) plates using sterile cotton swabs;

IV. Removing the mycelial growth of actinomycete strain R2 from YEME agar plates using sterile spatula;

V. Preparing the agar plugs with 5 mm diameter using sterile well cutter;

VI. Placing the agar plugs over the surface of each MHA plates seeded with test bacterial cultures;

VII. Incubating the MHA plates at 37° C. for 24 hours for bacteria and 48-72 hours for fungi;

VIII. Measuring the zone of inhibition of bacterial cultures around the agar plug and expressing in millimetre in diameter;

Table 1 provides the results of the antibacterial activity of the actinomycete strain by agar plug method.

TABLE 1

| | | Zone of inhibition |
|---|---|---|
| | | [expressed in |
| Sl. No. | Test cultures | millimetre in diameter] |
| 1. | Bacillus subtilis NCIM 2063 | 20 |
| 2. | Bacillus pumilus NCIM 2327 | 18 |
| 3. | Bacillus cereus NCIM 2106 | 23 |
| 4. | Staphylococcus aureus NCIM 2079 | 19 |
| 5. | Staphylococcus aureus (clinical) | 11 |
| 6. | Staphylococcus aureus (clinical; | 12 |
| | methicillin and vancomycin resistant) | |
| 7. | Bacillus subtilis MTCC | 10 |
| 8. | Acinetobacter baumanii (clinical; ESBL | 14 |
| | producing) | |
| 9. | Acinetobacter baumanii (clinical; ESBL | 15 |
| | producing) | |

### EXAMPLE 3

### Preparation of the Crude Extract of Compound of Formula (I)

Preparation of crude extracts of compound of formula (I) by agar plate culture:

The process of preparing the compound of formula (I) comprises following steps:

(i) inoculating a loopful of Actinomycetes strain R2 grown on yeast extract malt extract agar slants onto yeast extract malt extract agar plates (20 ml/plate) in 50 plates by continuous streaking;

(ii) incubating the agar plates at a temperature of 28° C. for a period of 7 days and obtaining mycelial growth;

(iii) removing the mycelial growth from the agar plates using a sterile spatula;

(iv) obtaining the agar medium containing the compound of formula (I);

(v) cutting the media into small pieces;

(vi) adding the agar media pieces into

(vii) adding the media pieces into beakers, wherein each beaker contains 100 ml of methanol as solvent;

(viii) incubating the beakers for a period of 24 hours at a temperature of 28° C. and extracting the compound of formula (I);

(ix) collecting the solvent portion and concentrating the same using rotary evaporator; and

(x) storing at 4° C.;

(xi) Quantifying the crude extract using electronic balance.

Each 100 ml quantity of methanol used as solvent provides approximately 40 mg of crude extract.

### EXAMPLE 4

### Antibacterial Activity of Crude Extract of the Actinomycete Strain

i. Preparing 10 mg/ml concentration of crude ethyl acetate extract of actinomycete strain R2 using ethyl acetate;

ii. Preparing crude extract discs by adding 10 μl of crude extract into each 5 mm diameter filter paper disc in order to get 100 μg/disc concentration;

iii. Drying the discs in laminar air flow cabinet;

iv. Testing the antibacterial activity of crude extract by disc diffusion method;

v. Preparing the cell suspensions of test bacterial cultures using nutrient broth and Sabouraud Dextrose, broth, respectively, and adjusting the turbidity to 0.5 McFarland standards;

vi. Inoculating the cell suspensions on Muller Hinton Agar (MHA) plates using sterile cotton swabs;

vii. Placing the crude extract impregnated paper discs over the surface of each MHA plates seeded with test bacterial cultures

viii. Incubating the MI-IA plates at 37° C. for 24 hours for bacteria;

ix. Measuring the zone of inhibition of bacterial cultures around the agar plug and expressing in millimetre in diameter;

Table 2 prOvides the results of the antibacterial activity of crude ethyl acetate extract of the actinomycete strain by disc diffusion method.

TABLE 2

| | | Zone of inhibition |
|---|---|---|
| | | [expressed in |
| S. No. | Test cultures | millimetre in diameter] |
| 1. | Bacillus subtilis NCIM 2063 | 20 |
| 2 | Bacillus pumilus NCIM 2327 | 21 |
| 3 | Bacillus cereus NCIM 2106 | 25 |
| 4 | Staphylococcus aureus NCIM 2079 | 20 |
| 5 | Staphylococcus aureus (clinical) | 14 |
| 6 | Staphylococcus aureus (clinical; | 18 |
| | methicillin and vancomycin resistant) | |
| 7 | Bacillus subtilis MTCC | 15 |
| 8 | Acinetobacter baumanii (clinical; ESBL | 18 |
| | producing) | |
| 9 | Acinetobacter baumanii (clinical; ESBL | 19 |
| | producing) | |

### EXAMPLE 5

### Purification of Compound of Formula (I):

A. Thin Layer Chromatography:

Purification of compounds were performed by preparative thin layer chromatography (TLC) using Merck silica gel 60 (GF254) pre coated aluminium (6x8 cm size) plates.

The crude pigment was purified by using preparative thin layer commercially available pre coated silica gel chromatography sheets (6×8 cm size) were used. To find out the best solvent system to separate the crude compound, the solvents were used in different proportions, among all solvent systems used, Ethylacetate: methanol (95:5) showed good separation.

The crude pigment was dissolved in 5 mL of ethyl acetate. With the help of capillary tube, the sample was spotted at the bottom of silica gel coated sheet (6×8 cm) and then it was placed in the developing 100 mL beaker containing mobile phase (Ethyl acetate/Methanol, 95:5) 5mL, covered with the watch glass in order to prevent the evaporation of the solvents. The solvent was allowed to run till it reached about half a centimetre below the top of the plate. After running, the 200 sheets were kept at room temperature for the complete drying of the plate. Spots on TLC were detected under UV light (254 and 365nm) and by spraying with concentrated H₂SO₄ followed, by heating at 105° C. for 5 min. After drying, the yellow pigment spot was scrapped, mixed with ethyl acetate and filtered using funnel fitted with whatman filter paper and Ethyl acetate was evaporated to dryness under vacuum to afford transitmycin as pure orange colour amorphous powder (1 0 mg). Rf value of the spot separated on the TLC plate was determined. The solvent system Ethyl acetate: methanol (95:5) was found to have good separation with single spot when compared to all the solvent systems used for TLC.

B. Column Chromatography

Column chromatography was carried out on Neutral Alumina (230-400 mesh) Column size: (id 30 m×90 cm)

The crude ethyl acetate extract (R2) was purified using column chromatography packed with neutral alumina using a gradient of 1% Methanol/Chloroform mixture (CH₃OH/CHCl₃) used as the eluent. Fractions were collected and concentrated under vacuum to afford Transitmycin as pure orange colour amorphous powder. The desired product was monitored in a TLC with pre coated alumina sheet silica. The Transitmycin (200 mg) was obtained as orange colour amorphous powder (Yield 20%), mp 240-242° C.

The purity, of Transitmycin was checked by Thin Layer chromatography with a solvent system 95:5 Ethylacetate/methanol. The compound had an Rf 0.8

### EXAMPLE 6

### HPLC Analysis of Crude Extract and Purified Compound of Formula (I)

Chromatographic Instrument and Conditions

HPLC analysis was carried out on a Shimadzu (Japan) RID-10A gradient high-performance liquid chromatographic instrument, equipped with twoLC-20AD pumps controlled by a CBM-10 inter-face module, Refractive index Detector RID 10A (Shimadzu) was used for the peak. Solvents were prefiltered by using a Millipore system and analysis was per-formed on a Luna 5u C₁₈(2) reversed-phase column, 100 (150×4.6 mm). The mobile phase was filtered through a 0.24 membrane filter and degassed by sonication before use. The analytical parameters were selected after screening a number of solvent systems and gradient fibres. Separation was achieved with /a two-pump gradient program for pump A (CH₃CN) and pump B (0.1% TFA in H₂O) as follows a linear gradient of acetonitrile and water from 20:80 to 50:50 in 20 minutes and then isocratic flow rate 1 ml/min 340 nm; The detection was at 340 nm. Injection size for sample was 20 μl column temperature was 30° C.

### EXAMPLE 7

### Characterisation of Purified Compound (I)

Solubility of purified compound is tested by adding the purified compound in 100 jxl of solvents such as methanol, chloroform, dichloromethane, diethyl ether, ethyl acetate and n-hexane. Melting point of the purified compound is tested using Tempo instrument and is determined as 240-242° C.

General Experimental Procedures.

Optical rotations were measured with a Autopol IV Automatic polarimeter, and the [α]_{D} values are given in deg cm² g⁻¹. UV spectra were recorded on a Jasco V 550 UV-VIS spectrophotometer. The UVₘₐₓ at 442 nm shows the presence of conjugated moiety. IR spectra were recorded on a Perkin Elmer spectrum one Fourier Transform Infrared spectrometer with KBr pellets. ‘H and ¹³C NMR spectra of Trasitymycin were recorded on a Bruker Avance 500 NMR spectrometer in CDCl₃ with TMS as internal Standard and with chemical shifts (8) reported in ppm. Two-dimensional 1H-1H COSY, DQF-COSY, NOESY, 1H-13C HSQC, HMBC, and spectra were recorded on a Bruker Avance 500 NMR spectrometer. MALDI-TOF MS analyses were performed using an Applied Biosystems ABI4700 TOF mass spectrometer in reflector mode with an accelerating voltage of 20 kV. HRESIMS were measured on a Q-TOF micro mass spectrometer (Waters USA) in Positive ion mode methanol as solvent. HPLC analysis was carried out on Luna 5u C₁₈ (2) 100 (150×4.6 mm) column with Shimadzu (Japan) RID-10A HPLC instrument, equipped with two LC-20AD pumps controlled by a CBM-10 inter-face module, Refractive index Detector. Marfey s method utilized a Waters Acquity UPLC coupled with a Thermo LCQ Deca XP MAX.QTOF-MS was recorded on an Agilent 6520-QTOF LCMS having a ESI source in Positive mode.

Preparative TLC was performed using Merck Si gel 60 F254 Precoated Aluminium sheets (20×20 cm). Analytical TLC was performed on the precoated aluminium TLC plates with silica gel 60 F254 (Merck, 0.25 mm) (normal-phase). Optical rotations at wavelengths 589 nm was measured with a 1.5 dm cell using an Autopol IV Automatic polarimeter and displayed as specific rotation (in deg cm3 g-1 dm-1 units). Optical measurements were obtained at a concentration of 2 mg/mL MeOH. CD spectrum was measured using methanol as solvent using JASCO J 815 CD spectrometer. LC-MS data were obtained using an API 3200 triple quadrupole MS (Applied Biosystems) equipped with a Shimadzu LC system.

HPLC analysis was carried out on a Shimadzu (Japan) RID-10A gradient high-performance liquid chromatographic instrument, equipped with twoLC-20AD pumps controlled by a CBM-10 inter-face module, Refractive index Detector RID 10A (Shimadzu) was used for the peak. Solvents were prefiltered by using a Millipore system and analysis was per-formed on a Luna 5u C₁₈ (2) reversed-phase column, 100 (150×4.6 mm).The mobile phase was filtered through 0.2μ membrane filter and degassed by sonication before use. The analytical parameters were selected after screening a number of solvent systems and gradient files. Separation was achieved with a two-pump gradient program for pump A (0.1% Acetic acid in CH₃CN) and pump B(0.1% Acetic acid in H₂O) as follows a linear gradient of acetonitrile and water'from 0:100 to 65:35 in 65 minutes flow rate 2 ml/min. The detection was at 254 nm, the absorption maxima close to all the compounds. Injection size for sample was 20 μl. column temperature was 30° C.

The compound is obtained as an orange coloured solid. The characteristics of the compound are as follows:

Colour: Orange colour amorphous powder

Yield: 200 mg, 20%

Mp.: 240-242° C.

[α]_{D}²⁵: −106′ (c=0.2, MeOH)

TLC:R_{f}−0.8(Ethyl acetate-Methanol, 95:5)

Solubility:Souble in Chloroform, Dichloromethane, Ethyl acetate, Methanol, Ethanol, Acetonitrile, DMSO, water. Insoluble in Hexane

UV: (MeOH)λ max(log ε)214(3.07), 240 (2.30), 4.25. (1.44), 442(1.51)nm

CD:[MeOH. [nm], (mdeg)]: λ_{?max} (Δε)195 (+11.1), 210(−21.0),242 (+4.7)

IR(KBr cm⁻¹),3435 cm⁻¹ for OH or NH,2958,2924 cm⁻¹,(m, —CH str, asym, CH₃ and CH₂), 2872 cm⁻¹,2853 cm⁻¹, (m, —CH str, sym, CH₃ and CH₂), 1746 cm⁻¹ (s, C═O str, Ester group), 1642 cm⁻¹, (s, —C═O str, 2° amide), 1524, 1503 (m, —NH bend, 2° amide), 1466 (m, CH bend (scissoring), CH₂), 1379 cm⁻¹ (s, —CH bend, isopropyl group), 1268 (s,C—O str, ester); 1099,1059,1017 (s,C—O of OH or NH), 720,712,694, 689 (s, —CH bend, oop, aromatic ring), 909 (w,CH₃ rocking).

H NMR (500 MHz, CDCk₃) (Table:1)

C NMR(125 MHz, CDCl₃): 179.0, 174.0,173.5,173.17,169.0, 168.8,167.5, 167.5,166.5,166.1, 165.9,144.34,145.93,145.04, 140.5, 132.19, 130.3,129.2,127.8,126.1,113.6,101.8,76.7,74.76,74.67,71.4, 71.2,58:5,57.2,56.4,54.9,54.7,54.3,51.3,29.6,29.6,29.3,22.6,29,21.6,19.2,19.2,19.09,19. 06,18.8, 17.14, 14.11, 7.77

HRESI-MS: m/z(pos.ions) 656.9243[M+2H]⁺², 1270.7069[M+H]⁺, 1291.8449[M+Na]⁺, 1307.9286[M+K]⁺

C₆₂H₈₄N₁₂O₁₇Na[M+Na]⁺ calc. 1291.5975, found.1291.8449

MALDI-TOF-MS:m/z(pos.ions) 1293.61316[M+Na+2H]⁺, 1309.93062 [M+K]⁺

m/z(neg.ions)1269.33344[M−H]⁻

C₆₂H₈₄N₁₂O₁₇Na[M+Na+2H]⁺ calc.1293.61950, found. 1291.61316 EI-MS:(70 ev) m/z (pos.ions)1348.1437,1291.4173[M+Na]⁺,

1224.7363,1191.8994, 1023.6241, 886.0243,743.2058,

614.8185,347.6111,202.5464,138.5079

LCESI-MS: m/z (pos.ions) 1291.5995[M+Na]⁺

C₆₂H₈₄N₁₂O₁₇Na[M+Na]⁺ calc.1291.5975, found.1291.5995

CHN:Anal.calcd for C₆₂H₈₄N₁₂O₁₇: C,58.66; H,6.67; N,13.24

Found: C,59.71,H,7.28; N,10.19

### EXAMPLE 8

### Chiral Amino Acid Analysis:

Transitmycin (3.0 mg) was dissolved in 6NHCl (1 mL) and heated in sealed glass tube at 110.0 for 24 h. The solvent was removed under reduced pressure, and the resulting material was subjected to further derivatization. The hydrolysate mixture (3 mg) or the amino acid standards (0.5 mg) were dissolved in 0.1 mL of water and treated with 0.2 mL of 1% 1-fluoro-2,4-dinitrophenyl-5-L-alaninamide (FDAA) (Marfey's reagent) in acetone (10 mg/mL in acetone) and 0.04 mL of 1.0 M sodium bicarbonate. The vials were heated at 50.0 for 90 min, and the contents after cooling at room temperature were neutralized with IN HCl. After degassing, an aliquot of the FDAA derivative was diluted in CH₃CN, Water (1:1) and analysed by reversed phase HPLC column Luna 5u C_{:8} (2) 100 (150×4.6 mm) and a linear gradient of acetonitrile and water containing 0.05% trifluoroacetic acid from 10:90 to 50:50 in 20 min and then isocratic. The flow rate was 1 mL/min, and the absorbance detection was at. 340 nm. The chromatogram was compared with those of amino acid standards treated in the same conditions.

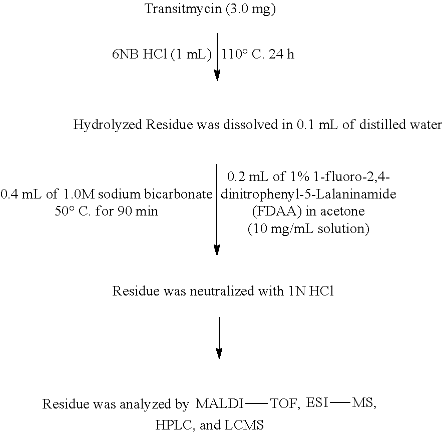

Analysis and characterization of the crude extract and purified compound of formula (I)

Assignment of Absolute Configuration amino acid in Transitmycin

Table 3 provides the results of analysis of L-FDAA derivatives of acid hydrolysate of Transitmycin by HPLC

TABLE 3

| | | | HPLC retention | |
|---|---|---|---|---|
| | HPLC retention times; | | times; Marfey's | |
| | Marfey's derivatives of | | derivatives of | |
| | standard amino acids | | Acid hydrolysate | |
| Amino acid | D | L | of Transitmycin | Assignment |
| Threonine | 15.682 | 13.987 | 14.021 | L |
| Proline | 17.035 | 16.519 | 16.520 & 17.055 | D & L |
| Valine | 21.244 | 19.248 | 19.252 | L |
| N-methyl | 22.089 | 20.814 | 21.263 | L |
| Valine | | | | |

### EXAMPLE 9

### Effect of Solvents on the Extraction of the Compound of Formula (I):

The process as elaborated in example 1 is carried our using solvents methanol, chloroform, dichloromethane, diethyl ether and ethyl acetate.

Table 5 gives the results of the effect of solvents on the extraction of the compound.

TABLE 5

| | Quantity of crude extract |
|---|---|
| Solvent extracts | (mg/100 ml) |
| Methanol | 40 |
| Chloroform | 41 |
| Dichloromethane | 40 |
| Diethyl ether | 10 |
| Ethyl acetate | 9 |

The results shows that the compound is extracted well in methanol, chloroform and dichloromethane compared to diethyl ether and ethyl acetate. Extracts in methanol, chloroform and dichloromethane gives better colour intensity as compared to the extract in diethyl ether and ethyl acetate. However, the extracts with methanol, chloroform and dichloromethane extracts shows presence of salt crystals and other debris. Ethyl acetate and diethylether extracts does not show any such salt crystals and debris.

### EXAMPLe 10

### Antimycobacterial Activity of Compound of Formula (I):

Stock Preparation:

Adding 10 mg of crude extract into 1 ml of 10% Dimethyl Sulfoxide (DMSO) and sterilizing the extract by filtration using 0.45μ filter.

Preparing Cell Suspension:

i. Adding standard strain Mycobacterium tuberculosis H37Rv growing on Lowenstein Jenson (LJ) slopes in to 5 ml of sterile glycerol 7H9 (G7H9) broth and mixing using vortex mixer for 2 minutes.

ii. Allowing the cell suspension to stand for few minutes for settling the clumps of bacteria.

Luciferase Reporter Phage (LRP) Assay:

i. Taking each 350 μl of G7H9 broth in seven cryo vials.

ii. Adding 50 μl of different solvent extracts into first five vials to give final concentration of 100 μg/ml.

iii. Adding 50 μl of 1% DMSO in to the sixth and seventh vials

iv. Adding 100 μl of M. tuberculosis H37Rv cell suspension in to all the vials.

v. Incubating all the vials at 37° C. for 72 hours.

vi. adding 50 μl of high titre phage phAE129 and 40 μl of 0.1M CaCl₂ into all the vials.

vii. Incubating all the vials at 37° C. for 4 hours.

viii. taking 100 μl of reaction mixture in cuvettes and adding D-luciferin.

ix. measuring relative light units (RLU) immediately in the kiminometer using 10 second integration time

x. Calculating the percentage of reduction in. RLU by using the following formula

$$\% RLU Reduction = \frac{Control RLU - Test RLU \times 100}{Control RLU}$$

Extracts resulting in more than 50% reduction in RLU are considered as active against M. tuberculosis.

Table 6 provides the results of the antimycobacterial activity of different solvent extracts

TABLE 6

| Solvent extracts | % reduction in RLU |
|---|---|
| Methanol | 58.31 |
| Chloroform | 18.07 |
| Dichloromethane | 22.71 |
| Ethyl acetate | 74.23 |
| Diethyl ether | 83.4 |

The results clearly indicate that among the different solvent extracts diethyl ether and ethyl acetate extract exhibits maximum activity.

The activity of the crude extracts is also tested on different strains of Mycobacterium tuberculosis.

Table 7 provides the results of the activity of crude extract on different strains of Mycobacterium tuberculosis.

TABLE 7

| Test organisms | % RLU reduction |
|---|---|
| M. tuberculosis H37Rv | 98.96 |
| M. tuberculosis SHRE sensitive | 98.46 |
| M. tuberculosis SHRE resistant | 97.49 |

The results clearly show that more than 95% RLU reduction is achieved. This indicates good activity against all the three M. tuberculosis strains tested.

### EXAMPLE 11

### Minimum Inhibitory Concentration of the Compound of Formula (I) against Mycobacterium tuberculosis:

The active fraction is dissolved in 1 ml of 10% DMSO (10 mg/ml) and is used as stock solution. Minimal inhibitory concentration of the purified fraction is tested at different concentration ranging from 50, 25, 12.5, 6.25, 3.125, 1.5 and 0.75 m/ml against standard strain M. tuberculosis H37Rv and clinical isolates of SHRE sensitive, multi drug resistant (MDR) and extensively drug resistant (XDR) Mycobacterium tuberculosis by LRP assay.

Table 8 provides the results for minimum inhibitory concentration (MIC) of the purified compound against different strains of Mycobacterium tuberculosis.

TABLE 8

| Organisms (strains) | MIC μg/ml) |
|---|---|
| M. tuberculosis H37Rv | <1 |
| M. tuberculosis (SHRE sensitive) | 1.5 |
| M. tuberculosis (SHRE resistant) | 6.25 |
| M. tuberculosis (XDR) | 6.25 |

The results clearly indicate that the compound is effective against all the strains of Mycobacterium tuberculosis. However, , the best activity is observed against Mycobacterium tuberculosis H37Rv.

### EXAMPLE 12

### Activity of Crude and Purified Compound of Formula (I) against Latent TB Bacilli

i. Preparing the stock solutions of crude extract and purified compound of formula (I) in 15 10% DMSO;

ii. Determining the inhibition of the growth of dormant tubercle bacilli grown under hypoxic condition according to Wayne's dormant model by the crude and purified compound of formula (I) at 10Cμg/ml and 10 μg/ml, respectively in sealed containers with moderate agitation;

iii. Finding the difference in the colony forming units before and after addition of crude and purified compound of formula (I).

Reduction in the CFU in M. tuberculosis cultures with purified compound of formula (I) is noticed in comparison with that of the CFU without the compound

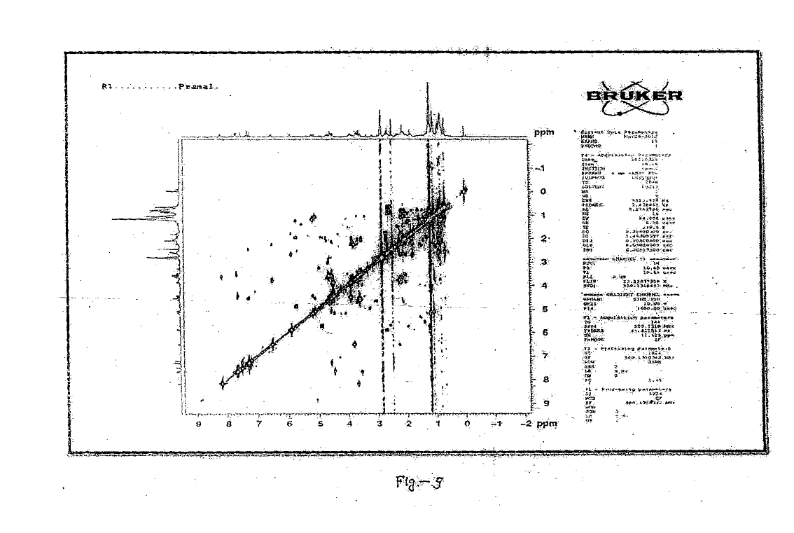

FIG. 9 provides the effect of crude extract and also the standard drugs INH and Rif against drug sensitive and MDR isolate of latent tubercle bacilli. INH was used as negative control and was resistant to dormant bacilli

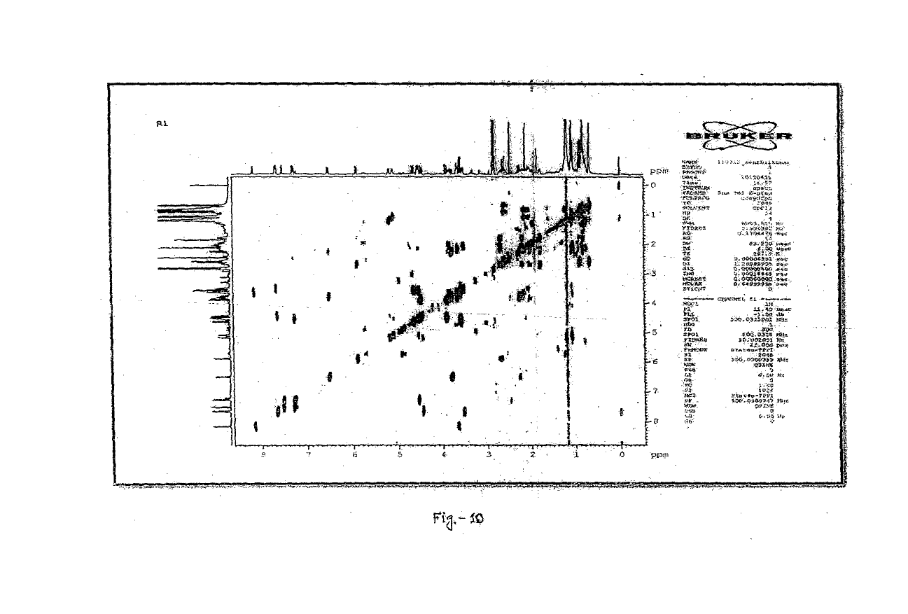

FIG. 10 provides the effect of purified compound of formula (I) and also the standard drugs INH and Rif against MDR and XDR isolate of latent tubercle bacilli.

### EXAMPLE 13

### Inhibitory Activity of Purified Compound of Formula (I) against MTB Biofilm

i. preparing the cell suspension of SHRE sensitive, MDR and XDR isolates of M. tuberculosis using 7H9 broth;

ii. developing the biofilm of M. tuberculosis isolates on 24 well tissue culture plates;

iii. adding 2 ml of Sautons medium (without Tween 80) and inoculating 20 μl of saturated planktonic culture of M. tuberculosis isolates;

iv. Adding 100 μg/ml of the compound of formula (I) in to the first wells, Rif and INH into the second and third wells, respectively;

v. Wrapping the plates with parafilm and incubating without shaking at 37° C. for 5 weeks in humidified conditions;

vi. Observing the plates for biofilm formation by M. tuberculosis isolates;

vii. Adding the purified compound of formula (I), Rif and INH into the 4^{th}, 5^{th} and 6^{th} wells containing the biofilms;

viii. Determining the viable counts of tubercle bacilli from the wells before and after adding the compound of formula (I), Rif and INH;

Biofilm formation is observed in the wells containing M. tuberculosis alone. In the wells containing M. tuberculosis cells and the compound of formula (I) there is no, biofilm formation.

CFU is determined at the end of 2 months and after the treatment of wells containing M. tuberculosis cells with compound of formula (I). There are no viable colonies found in the wells containing the compound, whereas the CFU determined before addition of the compound is 2×10⁶ /ml.

### EXAMPLE 14

### Minimum Inhibitory Concentration (MIC) of the Compound of Formula (I) against other Bacterial Pathogens

The minimum inhibitory concentration (MIC) of the compound of formula (I) is determined for other bacterial pathogens Staphylococcus aureus (NCIM5021), Pseudomonas aeruginosa (NClM5029) and Escherichia coli (NCIM2931). The minimum inhibitory concentration (MIC) is determined by micro dilution broth assay method with modifications using resazurin as an indicator as follows:

(i) dissolving the compound of formula (I) in absolute ethanol to a concentration of 10 mg/ml;

(ii) Serially diluting the compound and adding to successive wells in a 96 well microtiter plate and incubating with the bacterial pathogens for 18 hours at 37° C.;

(iii) maintaining the growth and sterility controls during the experiment;

(iv) adding 10 μl of 0.01% resazurin solution and incubating for 2 hours;

(v) visually assessing the color change.

Blue colour indicates inhibition of growth, indicating MIC.

The results of the activity are provided in table 9.

TABLE 9

| Organisms (strains) | MIC μg/ml) |
|---|---|
| Staphylococcus aureus (NCIM5021) | 138.88 |
| Escherichia coli (NCIM2931) | 17.36 |
| Pseudomonas aeruginosa (NCIM5029) | 17.36 |

The results clearly indicate that the active compound shows good activity against all the three bacterial pathogens tested.

This clearly establishes that the compound of the invention is not merely effective against Mycobacterium tuberculosis, but is also effective in controlling the growth of other bacterial pathogens. Therefore, the compound is also useful against other bacterial pathogens.

### EXAMPLE 15

### Anti-HIV Activity of Crude Extract and Compound of Formula (I)

Activity of Crude Extract:

i. Testing the in vitro antiviral activity of the crude extract on an infectious laboratory adapted subtype B strain of HIV-1;

ii. Infecting the activated healthy donor PBMC with 100TCID₅₀ of the virus per 1×10⁶ cells and cultured in the presence of varying concentrations of the crude extract (100 μg/ml, 50 μg/ml, 25 μg/ml and 10 μg/ml);

iii. Determining the HIV-1 p24 antigen production on day 7 as an indirect measure of viral replication in the culture supernatants using the Alliance HIV-1 p24 ELISA kit (Perkin Elmer, USA).

Viral inhibition is observed at all concentrations tested. Complete inhibition of growth of HIV virus is observed at all concentrations tested.

Activity of Purified Compound of Formula (I):

Virus production by transfection of 293T cells: 293T cells are plated at a concentration of 1×10⁶ cells/ml in a 100 mm culture dish and grown at 37° C. in a CO₂ incubator for 24 hours. Cells are transfected with 20 μg of HIV IIIB plasmid DNA using the mammalian cell transfection kit (Millipore). The culture supernatant is collected at 48 hours post transfection, clarified by centrifugation and stored in liquid nitrogen.

Titration of virus stock: Seven serial four-fold dilutions of virus stock, ranging from 1:16 to 1:65,635 are titrated in triplicate in a 96-well flat bottomed tissue culture plate containing 200,000 cells/well (PBMC stimulated with PHA for 72 hours). After 7 days of culture at 37° C. in a CO₂ incubator, the titration assay is terminated and the culture supernatants are tested for HIV-1 p24 antigen. The TCID50 (tissue culture infection dose50) is calculated employing the Spearman-Kaber method.

Testing for anti-HIV activity of compound: HIV IIIB is used as a representative Glade B virus and Indie-Cl as a representative Glade C virus. Healthy donor PBMC (Peripheral blood mononuclear cells) activated through PHA (Phyto heme agglutinin) stimulation for 72 hours are incubated with 100TCID₅₀ of the virus per 1×10⁶ cells for 2 hours at 37° C. The cells are washed twice to get rid of the unadsorbed virus and plated at a concentration of 200,000 cells/well in a 96-well tissue culture plate. Varying concentrations of the compound are added to triplicate wells (concentrations tested were 0.001 μg/ml, 001 μg/ml, 0.1 μg/ml, 1.0 μg/ml, 5.0 μg/ml and 10.0 tg/ml). Control cultures are set up without addition of the compound. Cultures are maintained for 7 days at 37° C. in a CO₂ incubator. On day 7, culture supernatants are tested for HIV-1 p24 antigen.

Measurement of HIV-1 p24 antigen: HIV-1 p24 antigen production is measured as an indirect measure of viral replication in the culture supernatants using the Alliance HIV-1 p24 ELISA kit (Perkin Elmer, USA).

Virus growth is .determined, by measuring p24 concentrations in culture supernatants. Table 10 below provides the results for the anti-HIV activity-of the compound.

TABLE 10

P24 antigen (pg/ml)

| Compound (μg/ml) | Clade B | Clade C |
|---|---|---|
| 0 | 2394 | 406 |
| 0.001 | 1603 | 390 |
| 0.01 | 337 | 310 |
| 0.1 | 163 | 344 |
| 1 | 144 | 302 |
| 5 | 147 | 295 |
| 10 | 163 | 296 |

Reduction in p24 levels indicates the level of inhibition. The results clearly indicate that the compound of the invention is effective against HIV.

Activity of purified compound of formula (I) against different eludes of HIV-1

I. Examining the activity of the compound of formula (I) on different HIV-1 subtypes;

II. The virus isolates tested were:

Subtype A: 92RW020

Subtype B: JR-FL

Subtype C: 92BR025

Subtype D: 92UG001

Subtype E: 92TH021

Subtype A/C: 92RW009

III. Infecting the activated donor PBMC with 100TCID₅₀ of primary clinical isolates representing different HIV-1 clades (clades A,B, C, D, E, A/E), as well as nevirapine resistant and AZT resistant strains, in the presence of purified compound of formula (I);

IV. Measuring the activity of the purified compound of formula (I) by measuring p24 antigen produced upon culture for 7 days;

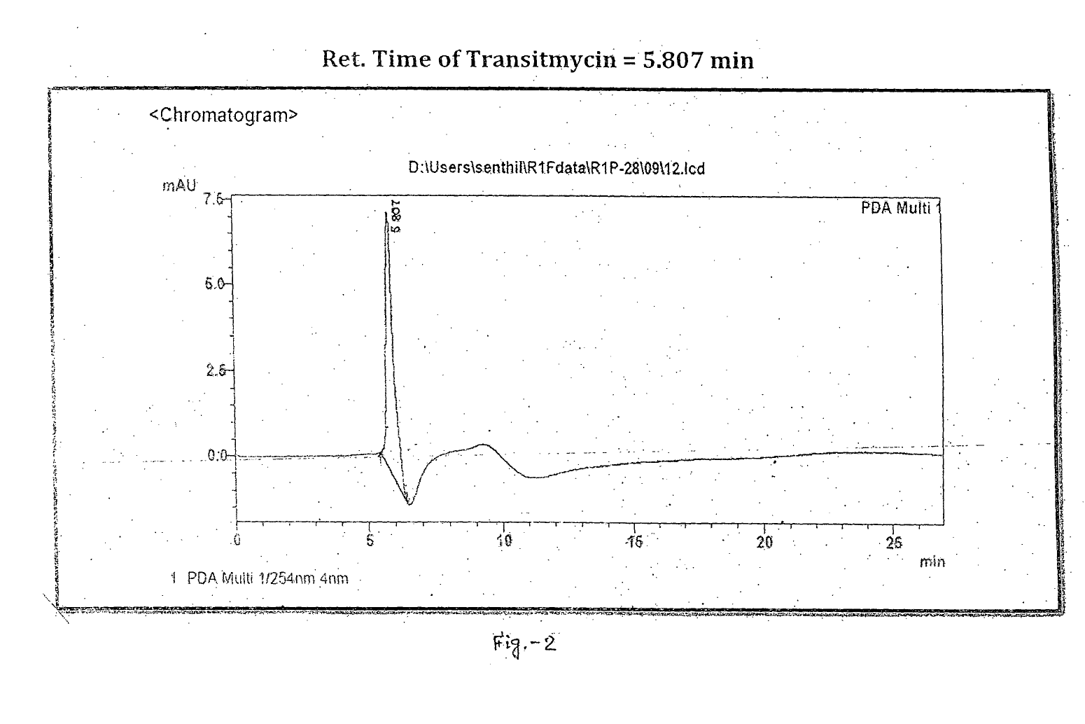

FIG. 2 provide the effect of the purified compound of formula (I) on various clades of HIV-1. The purified compound of formula (I) has activity on all the different strains of HIV-1 tested.

### EXAMPLE 16

### Cytotoxicity of the Compound of Formula (I):

Cytotoxicity of the compound is measured by adopting MTT assay (Mosmann, 1983) as follows:

(i) preparing, the sample by inoculating 3T3 cells in 5×10⁴ concentrations in each well of 96 well microtiter plates with in Dulbecco's modified Eagles medium (DMEM) containing 10% FBS, 100 U/ml Penicillin 100 g/ml Streptomycin;

(ii) incubating for a period of 2 days at a temperature of 37° C. in 5% CO₂ atmosphere (Astec Japan);

(iii) adding the compound of formula (I) in four different dilutions of 25, 50, 75, 100 μg/ml in DMSO to the medium and incubating the, cells for another 12 hours;

(iv) Discarding the growth medium in the plates and washing the wells with phosphate buffer saline (PBS);

(v) Adding MTT in growth medium at a final concentration of 0.5 mg/ml and incubating for 4 hours;

(vi) solublizing the insoluble formazan crystals' with 0.04N HCl in isopropylalcohol and measuring the absorption on a Spectramax Plus384 spectrophotometer (Molecular Devices, Calif., USA) at 570 nm.

For each of the samples evaluated, the test is performed in triplicate. The control cells are treated with PBS. Overnight experiment is done with DMSO alone as a control.

The results for cytotoxicity of the compound of formula (I) is given in table 11.

TABLE 11

| Concentration of the compound | Average % Viability | SD |
|---|---|---|
| Control | 100.00 | 0.00 |
| DMSO | 97.48 | 5.56 |
| 10M | 93.00 | 4.66 |
| 25M | 86.01 | 2.93 |
| 50M | 73.88 | 4.00 |
| 100M | 66.13 | 2.05 |

The results clearly show the viability of the cells for various concentrations of the compound. It is evident from the results that the compound of formula (I) shows very poor cytotoxic activity even at 100 M concentration.

### EXAMPLE 17

### Synthesis of Purified Compound of Formula (I) Predictable Derivatives—in Silico Approach

The in silico derivatives (n=27+251) of compound of formula (I) are subjected to QIKPROP module of SHROEDINGER software output.

TABLE 12
Table 12. The in silico derivatives of compound of formula (I) are subjected to
QIKPROP module of SHROEDINGER software output.

| | | | | | | | | | | Percent | | |
|---|---|---|---|---|---|---|---|---|---|---|---|---|
| | | | | | QP | | | | Human | Human | Rule | Rule |
| | mol | | | | log | QPP | Q | | Oral | Oral | Of | Of |
| molecule | MW | volume | donorHB | accptBB | Poct | Caco | PlogBB | metabolism | absorption | absorption | Five | Three |
| Methylketone | 377.609 | 1486.747 | 0 | 4 | 15.361 | 597.221 | −1.963 | 7 | 1 | 100 | 1 | 2 |
| isoprophone | | | | | | | | | | | | |
| compound of | | | | | | | | | | | | |
| formula (I) | | | | | | | | | | | | |
| Allyl Isoprepyl | 375.637 | 1520.708 | 0 | 2 | 14.322 | 1601.713 | −1.55 | 7 | 1 | 100 | 1 | 2 |
| compound of | | | | | | | | | | | | |
| formula (I) | | | | | | | | | | | | |
| Methylketone | 363.582 | 1449.083 | 0 | 4 | 15.071 | 580.196 | −1.981 | 6 | 1 | 100 | 1 | 1 |
| ethyl compound | | | | | | | | | | | | |
| of formula (I) | | | | | | | | | | | | |
| Allyl ethyl | 361.61 | 1472.491 | 0 | 2 | 13.624 | 1575 | −1.561 | 6 | 1 | 100 | 1 | 1 |
| compound of | | | | | | | | | | | | |
| formula (I) | | | | | | | | | | | | |
| Methylketone | 365.63 | 1425.302 | 2 | 4 | 17.1 | 482.337 | −2.098 | 6 | 1 | 94.058 | 1 | 0 |
| Silicon | | | | | | | | | | | | |
| compound of | | | | | | | | | | | | |
| formula (I) | | | | | | | | | | | | |
| Allyl Silicon | 363.657 | 1463.343 | 2 | 2 | 16.5 | 1321.067 | −1.716 | 6 | 1 | 100 | 1 | 1 |
| compound of | 386.414 | 1371.601 | 0 | 2 | 12.746 | 1436.25 | −1.362 | 4 | 1 | 100 | 1 | 1 |
| formula (I) | | | | | | | | | | | | |

QikProp's use of whole-molecule descriptors that have a straightforward physical interpretation (as opposed to fragment-based descriptors) could provide a useful pathway for medicinal chemists to modify ADME properties. QikProp has been thoroughly evaluated at many major pharmaceutical companies and found to be extremely useful in the context of both high-throughput library screening and lead optimization.

Schrödinger's QikProp is an extremely fast ADME properties prediction program. It provides the following benefits:
Wide range of predicted properties: QikProp predicts the widest variety of pharmaceutically relevant properties—octanol/water and water/gas log Ps, log S, log BB, overall CNS activity, Caco-2 and MDCK cell permeabilities, human oral absorption, log Khsa for human serum albumin binding, and log. 1050 for HERE K+-channel blockage -so that decisions about a molecule's suitability can be made based on a thorough analysis.
Lipinski Rule-of-Five and Jorgensen Rule-of-Three: QikProp has the ability to check for Lipinski Rule-of-Five and Jorgensen Rule-of-Three violations to provide an at-a-glance measure of whether a compound is drug-like.
Lead generation: QikProp rapidly screens compound libraries for hits. QikProp identifies molecules with computed properties that fall outside the normal range of known drugs, making it simple to filter out candidates with unsuitable ADME properties.

These shortlisted derivatives can be synthesized to optimize lead compound from compound of formula (I)
Improving accuracy: QikProp computes over twenty physical descriptors, which can be used to improve predictions by fitting to additional or proprietary experimental data, and to generate alternate QSAR models.

The 27 and 251 derivatives were narrowed down to 6 due to the ADME filters, the results depicted in the above Table enabled the selection of six possible derivatives of compound of formula (I).

The druglikeliness of the six derivatives of compound of formula (I) based on the ADME results of QIKPROP validates its claim for synthesis.

Advantages of the Invention:

1. The compound of the invention is effective against multiple drug resistant and extensive drug resistant strains of Mycobacterium tuberculosis.

2. The compound of the invention is also effective against other bacterial pathogens.

3. The compound of the invention is effective against Human Immuno Deficiency Virus (HIV).

4. The process of producing the compound of the invention is a simple process and does not require complex laboratory set-up. Therefore, the process of production of compound is economically viable.

5. The compound of the invention is a natural product. Also, the compound is produced through naturally occurring microorganisms. Therefore, the compound itself or the process of producing the same are eco-friendly and does not pose any threat to environment.

6. The compound shows very poor cytotoxic activity. Therefore, the compound can be effectively used to manufacture pharmaceutical formulations against bacterial and viral pathogens.

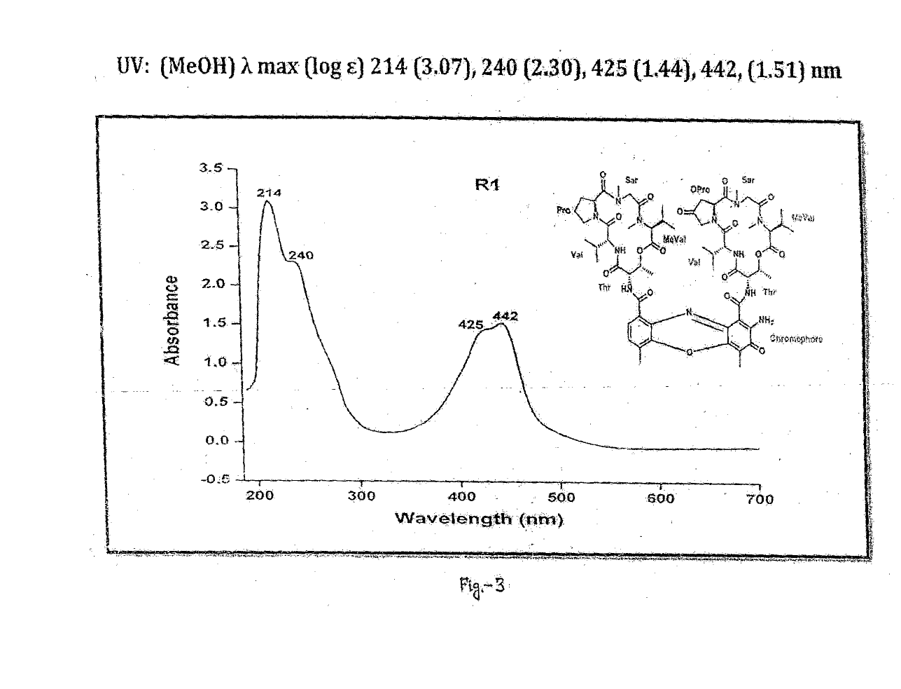

Transitmycin (1) was isolated as a orange colour amorphous powder with [α]_{D}²⁵: −106° (c=0.2, MeOH). The molecular formula was established as C₆₂H₈₄N₁₂O₁₇ by Positive HRESI-MS mass spectrum, showing protonated pseudo molecular ion peak [M+H]⁺ at m/z 1270.7069, showed intense peaks, due to Na and K adducts respectively, at m/z 1291.8307 [M+Na]⁺ and 1307.8124. [M+K]⁺ (Calcd. For C₆₂N₈₄N₁₂NaO₁₇. 1291.5975: Found: 1291.8307). Similarly from MALDI TOF MS spectrum transitmycin showed intense peak in positive mode at m/z 1293.61316[M+Na+2H^{]+3}, at m/z 1309.93062[M+K]⁺ and in negative mode at m/z 1269.33344[M−H]⁻. The ¹H and ¹³C NMR spectra exhibited the typical features of two penta peptido lactone ring attached with phenoxazinone chromophore, i.e., each ring contains four amide carbonyl resonances and one ester carbonyl in one ring (δC179.0, 174.0, 173.5, 173.1, 169.02, 198.8, 167.5, 166.5, 166.56, 166.3, 166.1, 165.9), together with phenoxazinone chromophore (:147.3, 145.9, 145.0, 140.5, 132.1, 130.3, 129.2, 127.8, 126.1, 113.6, 101.8) and one the amino group contains keto group at 208.8 in the ¹³C NMR spectrum and four amide proton signals (δH 8.2, 7.74,7.69, 7.2) and four N-methyl group at (δH 2.94,2.92, 2.90, 2.89) in the 1H NMR spectrum (Table Q. In addition, the ID NMR spectra of 1 indicated the presence of eight methyl's due to four isopropyl groups, The UV/vis absorption spectra with maximal absorbance at 240 nm and 442 nm support the presence of an aminophenoxazinone chromophore in their structure. From 1 H-1 H COSY and TOCSY experiments, five amino acid systems of Pro, Thr, Val, N-methyl val, and Ser were determined. The assignments of the protonated carbons were obtained from the HSQC spectrum, in combination with inspection of the HMBC spectrum. By comparison of the UV. spectrum (λmax 442 nm, in MeOH) of transitmycin with that of actinomycin series (Amax 440 nm, in MeOH), it was suggested that the contained an aminophenoxazinone chromophore residue. In ¹H NMR two ortho coupled protons at. 7-H 7.59 and 7.28 of a 1,2,3,4-tetrasubstituted aromatic ring, and two 3H singlets at 6-H, 2.4 and 4-H,1.95 of methyl groups in peri-position of an aromatic system. This is characteristic for the phenoxazinone chromophore (FIG. 3) in various actinomycins. The result was further confirmed by the HMBC correlations between the 8-H (SH-7.59) of the tetrasubstitued double bond and the carbonyl resonances at 6C 166.06. The carbonyl carbons of Pro, Thr, Sar, Val, and N-methyl Val, were clearly assigned to 8C (SC 179.0, 174.0, 173.1, 169.02, 498.8, 167.5: 166.5, 166.56, 166.3, 166.1,165.9) on the basis of, the observed correlations between carbonyl groups protons of the same amino acid residue in the HMBC spectrum.

All residues were connected on the basis of HMBC and’ NOESY correlations, thus establishing the amino acid sequences and overall constitution.

Detailed analysis of ‘H, ¹³C, ¹H-¹H COSY, HSQC and HMBC NMR spectra revealed ten amino acids for Transitmycin, which being identical with those of actinomycin X₂ (2 X MeVal, 2 X Thr, 2 X Sar, 2 X Val, Proline and keto proline). OPro was easily identified by the ketone moiety (SC 208.6) and the altered chemical shifts and coupling patterns of the neighbouring methylene groups.

The absolute configurations of the amino acids were supposed to be identical with actinomycin X₂, as indicated by the negative optical rotation values and the strong cotton effect at about 210 nm in the CD spectra. The assignment of the amino acids was done primarily by analysis of the HSQC and 1H1H-COSYcorrelations and completed by an HMBS spectrum. Additionally, a small amount of 1 was hydrolyzed, and the free amino acids were analyzed by HPLC after chiral derivatization with Marfey's reagent. Comparison with authentic standards revealed the presence of L-MeVal, L-Thr, L-Proline and Valm as L-Valine one of the Proline as D. Hence, we named the unusual, newly found compounds Transitmycin as X-type members.

1. A compound represented by formula (I) (Transitmycin) 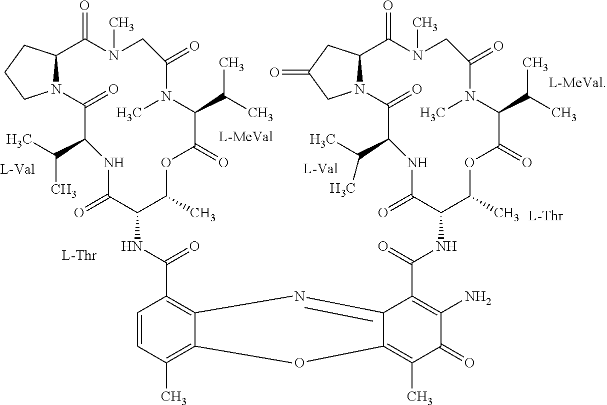 [SMILES: Cc1c2oc3c(C)ccc(C(=O)N[C@@H]4C(=O)N[C@@H](C(C)C)C(=O)N5CCC[C@H]5C(=O)N(C)CC(=O)N(C)[C@@H](C(C)C)C(=O)O[C@@H]4C)c3nc-2c(C(=O)N[C@@H]2C(=O)N[C@@H](C(C)C)C(=O)N3CC(=O)C[C@H]3C(=O)N(C)CC(=O)N(C)[C@@H](C(C)C)C(=O)O[C@@H]2C)c(N)c1=O]
wherein the compound is effective against bacterial and viral pathogens.

2. The compound as claimed in claim 1, wherein the compound is effective against Mycobacterium tuberculosis, Bacillus subtilis, Bacillus pumilus, Bacillus cereus, Staphylococcus aureus, and Acinetobacter baumanii.

3. The compound as claimed in claim 2, wherein the compound is effective against multiple drug resistant and extensively drug resistant strains of Mycobacterium tuberculosis.

4. The compound as claimed in claim 2, wherein the compound is effective against Streptomycin, Isoniazid, Rifampicin, and Ethambutol (SHRE) sensitive and SHRE resistant strains of Mycobacterium tuberculosis.

5. The compound as claimed in claim 1, wherein the compound is effective against Human Immunodeficiency Virus (HIV).

6. A composition comprising the compound of claim 1 in an amount of 0.1 to 5C μg/ml along with pharmaceutically acceptable additives, excipients and adjuvants.

7. The composition as claimed in claim 6, wherein the composition is formulated as one or more of a liquid, solid, powder and lozenge.

8. A process of preparing a compound represented by Formula (I), said process comprising the steps of:
(i) inoculating Actinomycetes strain MTCC 5597 onto a suitable agar based media plate;
(ii) incubating the agar plate(s)plate at a temperature of 20° C. to 40° C. for a period of 3 to 10 days and obtaining mycelial growth;
(iii) removing the mycelial growth from the agar plates plate and obtaining the agar medium containing the compound of formula (I);
(iv) optionally cutting the media into pieces;
(v) adding the media pieces into a suitable solvent;
(vi) incubating the media dissolved in solvent at a temperature of 23° C. to 30° C. for a period of 3 to 18 hours and extracting the compound of formula (i);
(vii) collecting the solvent part and concentrating the same to form a concentrate;
(viii) obtaining the concentrate containing the compound of formula (I); and
(ix) purifying the compound.

9. The process as claimed in claim 8, wherein the media is selected from the group consisting of yeast extract malt extract agar, glycerol asparagine agar, oatmeal agar, czapek's dox agar, and tyrosine agar.

10. The process as claimed in claim 8, wherein the agar plate is incubated at a temperature of 28° C.

11. The process as claimed in claim 8, wherein the agar plate is incubated for a period of 7 days.

12. The process as claimed in claim 8, wherein the media dissolved in solvent is incubated at a temperature of 28° C.

13. The process as claimed in claim 8, wherein the media dissolved in solvent is incubated for a period of 24 hours.

14. The process as claimed in claim 8, wherein the concentrate containing the compound of formula (I) is stored at a temperature of 4° C. to 25° C. before purification.

15. The process as claimed in claim 14, wherein the concentrate is stored at a temperature of 4° C.

16. (canceled)

17. A kit comprising a compound of formula (I) as claimed in claim 1 and an instruction manual.

18. (canceled)

19. A method of treating a disease caused by one or more of a bacterial or viral pathogen comprising administering to a patient in need thereof a composition comprising the compound of Formula (I) in an amount effective to reduce or eliminate the bacterial or viral pathogen wherein the bacterial or viral pathogen, is selected from the group consisting of Mycobacterium tuberculosis, Bacillus subtilis, Bacillus pumilus, Bacillus cereus, Staphylococcus aureus, and Acinetobacter baumand Human Immunodefidiency Virus (HIV).

20. (canceled)
